(19) United States
(12) Patent Application Publication
(10) Pub. No.: US 2007/0264281 A1
(43) Pub. Date: Nov. 15, 2007

(54) Vaccination Vectors Derived From Lymphotropic Human Herpes Viruses 6 and 7

(76) Inventor: Niza Frenkel, Tel-Aviv (IL)

Correspondence Address: NATH & ASSOCIATES, 112 South West Street, Alexandria, VA, 22314, US

(21) Appl. No.: 10/567,064

(22) PCT Filed: Aug. 4, 2004
(86) PCT No.: PCT/IL04/00719

§ 371 Date: Aug. 23, 2006

Related U.S. Application Data
(60) Provisional application No. 60/491,978, filed on Aug. 4, 2003.

(51) Int. Cl. A61K 39/12; C12N 15/00; C12N 5/00; C12N 7/00; C12P 21/00

(52) U.S. Cl. 424/199.1; 435/235.1; 435/320.1; 435/325; 435/69.1

(57) ABSTRACT

A vector is presented comprising a DNA sequence derived from HHV-6 or HHV-7, said DNA sequence comprising an origin of replication, a cleavage and packaging signal and a promoter sequence which induces expression of at least one nucleic acid sequence product in a lymphocyte cell host, wherein administration of said DNA vector to a mammal results in an immune response in said mammal. Optionally the vector comprises at least one foreign nucleic acid sequence which elcits the immune response. Also presented are cells comprising the vectors, and concatameric DNA vector produced using such vectors. In addition, methods for the production and use of the above are discribed, including pharmaceutical composiotions comprising same.

### FIELD OF THE INVENTION

The present invention is generally in the field of viral vectors as vaccination vectors.

### BACKGROUND OF THE INVENTION

### Prior Art

The following are references considered to be relevant for the subsequent description:
Ablashi D, A. H., Balachandran, N., Josephs, S. F., Hung, C. L., Krueger G. R., Kramarsky, B., Salahuddin S. Z. and Gallo R. C. (1991). Genomic polymorphism, growth properties, and immunologic variations in human herpesvirus-6 isolates. Virology 184:545-552.
Borenstein, R., Singer, O., Mosen A. and Frenkel N. (2004). Use of an amplicon-6 vector derived from Human Herpesvirus 6 for efficient expression of membrane-associated and secreted proteins in T cells, J. Virol. 78:4730-4743.
Engels, B., H. Cam, T. Schuler, S. Indraccolo, M. Gladow, C. Baum, T. Blankenstein, and W. Uckert. 2003. Retroviral vectors for high-level transgene expression in T lymphocytes. Hum Gene Ther 14:1155-68.
Frenkel, N. and Roffman, E. (1996) Human herpesvirus 7. Virology. Third Edition. Eds. B. N. Fields, D. Knipe, P. M. Howley et al. Lippincott, Raven Press, pp. 2609-2633.
Frenkel, N., E. C. Schirmer, Wyatt L,. S. Katsafanas G, Roffman, E., Danovich R. M. and June, C. H. (1990) Isolation of a new herpesvirus from human CD4+ T cells. Proc. Natl. Acad. Sci. USA. 87:748-752.
Frenkel, N., Katsafanas, G. C., Wyatt, L. S., Yoshikawa, T., and Asano, Y. (1994). Bone marrow transplant recipients harbor the B variant of human herpesvirus 6. Bone Marrow Transplant 14:839-43.
Frenkel, N., Singer, O. and Kwong, A D. (1994) The herpes simplex virus amplicon—a versatile defective virus vector. Gene Therapy. 1:540-546.
Hanke T., Bamfield C., Wee E. G., Agren L., Samuel R. V., Larke N., Liljestrom P. (2003). Construction and immunogenicity in a prime-boost regimen of a Semliki Forest virus-vectored experimental HIV clade A vaccine. J Gen Virol. 84:361-368.
Meeker, T. C., L. T. Lay, J. M. Wroblewski, F. Turturro, Z. Li, and P. Seth. 1997. Adenoviral vectors efficiently target cell lines derived from selected lymphocytic malignancies, including anaplastic large cell lymphoma and Hodgkin's disease. Clin Cancer Res 3:357-64.
Katsafanas, G. C., Schirmer, E. C., Wyatt, L. S. and Frenkel, N. (1996) In vitro activation of human herpesviruses 6 and 7 from latency. Proc. Natl. Acad. Sci. 93: 9788-9792.
Taylor-Papadimitriou J, Burchell J, Miles D W, Dalziel M. (1999) MUC1 and cancer. Biochim Biophys Acta. 1455(2-3):301-13.
Pellett, D. (2002). Human Herpesviruses 6A, 6B, and 7 and their replication. Virology, Volume 2, Chapter 80, pp. 2769-2784. Lippincott-Raven publishers.
Rapaport, D., Engelhard, D., Tagger, G., Or, R., and Frenkel, N. (2002). Antiviral prophylaxis may prevent human herpesvirus-6 reactivation in bone marrow transplant recipients. Transpl Infect Dis 4:10-6.
Romi H., Singer O., Rapaport D., and Frenkel N. (1999), Tamplicon-7, a novel T-lymphotropic vector derived from human herpervirus 7. J. Virol. 73:7001-7.
Schirmer E. C., Wyatt L. S., Yamanishi K., Rodriguez, W. J., and Frenkel N. (1991) Differentiation between two distinct classes of viruses now classified as human herpes virus 6. Proc. Natl. Acad. Sci., USA, 88:199-208.
U.S. Pat. No. 5,230,997 Methods of detecting the presence of human herpesvirus-7 infection.
U.S. Pat. No. 6,503,752, Lymphotropic agents and vectors.
U.S. Pat. No. 6,544,780, Adenovirus vector with multiple expression cassettes.
WO 99/07869, Live recombinant vaccine comprising inefficiently or non-replicating virus.
Wyatt L., and Frenkel N. (1992) Human herpes virus 7 is a constitutive inhabitant of adult human saliva. J. Virol. 66:3206-3209.
Yamanishi, K. (1992). Human herpesvirus 6. Microbiol Immunol 36:551-61.

Human herpes virus-6 (HHV-6) was first isolated from peripheral blood mononucleur cells (BMC) of patients with lympho-proliforative disorders as well as from patients suffering from acquired immune deficiency syndrome (AIDS).

Two types of HHV-6 strains are recognized today and designated as variant A and variant B. They are closely related variants having DNA sequence homology ranging from 75 to 97%, depending on the gene(s). They differ in their growth properties, restriction enzyme patterns and antigenicity and they are also distinct epidemiologically (Pellet, 2002, Schirmer et al. 1991, Ablashi et al., 1991). Only the HHV-6B variant appears to be associated with human diseases. It infects the majority of children during the first 2 years of life. The virus causes roseola infantum or Exanthem Subitum (ES), usually a mild disease, characterized by several days of spiky fever and skin rash (Yamanishi et al., 1988). In some ES patients, the disease can extend to the central nervous system (CNS), up to fatal fulminate hepatitis. Furthermore, reactivation of HHV-6B from latency could play a role in some post-transplant complications, especially in patients with impaired immune capabilities, including AIDS patients and patients receiving preparatory immunosuppressive therapy in bone marrow transplantation (BMT) (Rapaport et al., 2002). The HHV-6B reactivation can cause late engraftment, up to lethal encephalitis.

In contrast to disease association of HHV-6B, symptomatic HHV-6A infections in children are rather rare and the virus is not known to be associated with children's diseases or in reactivation from latency in transplanted patients (Frenkel et al., 1994, Schirmer et al., 1991).

HHV-6 employs CD46 as a cellular receptor for entry into a wide range of cells, including mature T lymphocytes, lymph nodes, macrophages, monocytes, dendritic cells, kidney tubule endothelial cells, salivary glands, as well as CNS.

Human herpes virus-7 (HHV-7) is a DNA virus first isolated in the laboratory of the inventor of the present invention from activated T cells expressing the CD4 antigen (see U.S. Pat. No. 5,230,997, Romi et al. 1999, Frenkel et al., 1990). Cells expressing this antigen on their membrane will hereinafter be referred to as “CD4⁺ cells. The HHV-7 virus uses CD4 as an entry receptor.

HHV-7 was found to be distinct, both molecularly and antigenically, from all previously identified herpes viruses. HHV-7 replicates well in lymphocytes and particularly in T cells including CD4⁺ T cells and possibly other cells carrying the CD4 marker.

HHV-7 can persistently infect salivary glands, and it is continuously secreted into the saliva of more than 95% of humans (Wyatt and Frenkel, 1992). Although the virus infects the majority of children in early childhood, no known disease is associated with the virus. Latent virus genomes can be identified in many healthy individuals, and the virus can be activated from latency in vitro, by exposing T cells to activation conditions (Katsafanas et al., 1996). No HIV -7 reactivation has been reported in bone marrow transplantation (Rappaport et al., 2002).

The HHV-6A, HHV-6B and HHV-7 genomes are linear, double-stranded DNA molecules of 162-170 Kb. The genomes are composed of a 143 Kb segment of unique (U) sequences, bracketed by direct repeats DR_{L} (left) and DR_{R} (right), (Pellet et al. 2002). The viral genomes have similar arrangement of genes across the genomes (Pellet 2002.). HHV 6A, 6B and 7 each have a single DNA replication origin (oriLyt) (Dewhurst 1993; Romi et al., 1999; Pellet 2002) which replicates in the nucleus by the rolling circle mechanism, as shown by group (Romi et al, 1999). The DR sequences are bound by the pac-1 and pac-2 herpes virus conserved packaging signals (Frenkel and Roffman, 1996). The genome circularizes prior to the rolling circle replication, which leads to the formation of a complete pac-1-pac-2 cleavage/packaging signal. The consequent rolling circle replication generates large concatameric molecules, with pac-1-pac-2 signals bounding the repeats. The HSV amplicons, amplicon-6 and Tamplicon-7 vectors derived from HSV-1, HHV-6 and HHV-7, respectively, were previously described (U.S. Pat. No. 6,503,752). The constructed vectors contain a viral DNA replication origin, cleavage and packaging signals and the transgene(s). In the presence of helper virus functions the amplicon plasmid is replicated by the rolling circle mechanism and generates huge concatameric genomes which can be cleaved between the pac-1 and pac-2 signals. The most efficient cleavage occurs when the DNA molecules reach approximately full length 135-150 Kb genomes, made of identical amplicon repeats. The packaged amplicons are replication defective, but can enter into new cells and express their transgenes at high efficiency, due to sequence reiteration.

During packaging the concatamers are cleaved and packaged at 29-35 bp from pac-2, and 41-46 bp from pac-1 signals (Frenkel and Roffman, 1996; Romi et al., 1999). This process is most efficient for full-length DNA genomes (i.e. 135-150 kb). The capsids acquire the tegument layer in intra-nuclear tegusome structures, after which the particles appear to be released into the cytoplasm via fusion with the nuclear membrane. Envelopment occurs by budding into cytoplasmic vacuoles, which then fuse with the cell membrane to release mature particles (Roffman et al., 1990). The pac-1 and pac-2 signals are necessary for the entry of the packaged DNA into the cytoplasm and for further exit out of the cells and into the medium. The rolling circle mechanism and consequent cleavage and packaging processes are utilized in the production of the defective virus amplicon vectors.

Vaccinations have traditionally included injecting into the body an attenuated or killed form of a bacterium or virus, or injection of denatured proteins. While efficient in many cases, this form of vaccination is not effective in other cases, such as integral membrane proteins, HIV-related proteins, etc. Furthermore, such vaccines raise concerns regarding the ability of a live virus to establish latency, to reactivate, and to recombine with virulent wild type viruses, in addition to concerns regarding the oncogenic potential of some viral genes.

Another approach is DNA vaccination, whereby the DNA that encodes the desired protein to which immunity is sought is injected into the body, usually as part of a plasmid.

Another vaccination approach - genetic vaccination, involves mutant viruses which do not cause disease and which serve as vectors for introducing a cargo gene of interest into the host's cells. The gene is translated and expressed by the cells, and the protein product may induce an immune response in the host. Viruses are more efficient as vaccination vehicles than plain DNA since they enter host cells efficiently, and may also replicate in the cells thereby increasing the level of expression of their cargo gene.

Genetic vaccination has been described using the Vaccinia virus and mutants thereof, mainly the modified Vaccinia Virus Ankara (MVA, WO9907869) and the Adenovirus (U.S. Pat. No. 6,544,780). Unfortunately, Vaccinia has been shown to cause complications in individuals who were previously vaccinated against smallpox, and immune memory in individuals who have previously received Vaccinia virus may prevent recognition of any foreign gene insert (McDermott et al., 1989).

Hanke et al. (2003) describe a human immunodeficiency virus (HIV) vaccine that consists of Semliki Forest Virus (SFV), and a cargo gene encoding HIVA, which is an immunogen derived from HIV-1 clade A. In the mouse, the SFV.HIVA vaccine induced T cell-mediated immune responses and induced T cell memory that lasted for at least 6 months. However, SFV.HIVA is even less immunogenic than modified Vaccinia Virus Ankara carrying HIVA (MVA.HIVA).

Several groups have reported that lymphocytes could not be efficiently transduced employing adenoviral vectors. In their studies of 33 different lymphocytic cell lines Meeker and coworkers (1997) used adenovirus vectors carrying the β-gal marker and found that only limited number of cell lines had significant fluorescence, whereas the majority of tested cell lines had low expression efficiency. Five different T cell lines tested showed almost no expression.

Retroviral vectors, such as Moloney Murine Leukemia Virus (Mo-MLV), are commonly used to express genes in T lymphocytes. However, here also, the expression levels are often unsatisfactory . Significantly improved vectors have been recently described by Engels and coworkers (Engel., 2003). However, retroviral vectors might have disadvantages due to their integration into the host chromosomes, which might cause hazardous disruption or activation of host gene expression.

Dendritic cells (DCs) are efficient antigen-presenting cells (APCs) eliciting strong proliferative response of T lymphocytes to antigens and to recall proteins. In general they activate the immune response by capturing antigens in peripheral tissues and migrating to secondary lymphoid organs where they sensitize naive T lymphocytes to the antigen. Mature dendiritic cells express high levels of Major Histocompatibility complexes (MHC) class II and co-stimulatory molecules on their surface thereby acquiring the ability to prime CD4+ T lymphocytes. Treatment of the DS with tumor necrosis factor (TNF) receptor triggers their transition from immature to mature antigen presenting DCs.

Mucin MUC1 is a large, transmembrane glycoprotein localized normally to the apical membrane of normal epithelial tissues (Taylor-Papadimitriou et al., 1999). The MUC1 protein extends above the cell surface; it has a high level of sialic acid and is negatively charged. The extra cellular domain is made up largely of 20 amino acid tandem repeats (TRs). The number of repeats differs in different alleles. MUCI was reported to serve as ligand for ICAM5 expressed by endothelial cells. This adhesion molecule is known to be involved in recruiting macrophages into tumor site.

Aberrant expression of the MUC1 protein is observed in different carcinomas, including prostate, lung, breast ovarian, pancreas, renal and certain heamtopoeitic neoplasms. The protein is a recognized tumor antigen immunogenic in humans and isolated cytotoxic T cells (CTL) from breast cancer and ovarian cancer patients were found to kill MUC 1 expressing cells in a non-HLA restricted fashion.

Glossary

Vector refers to a DNA molecule capable of carrying a foreign nucleic acid sequence of interest (see below). This includes the DNA vector per se, as well as the vector that is packaged in a virion particle. The amplicon vector includes an origin of replication, a promoter sequence which allows expression in a host and a cleavage and packaging signal.

Concatameric vector means a DNA molecule comprising two or more repeats of at least one vector.

Lymphotropic Vector refers to a vector that is specifically capable of being expressed in lymphatic cells. This includes the DNA vector per se, as well as the vector that is packaged in a virion particle, in which case the targeting of the vector will be more efficient in blood cells which include T cells, B cells, monocytes, macrophages, NK cytotoxic T cells (CTL) and dendritic cells. When the lymphotropic vector is amplicon-6, it is also capable of being targeted to and expressed in other cells of non-lymphatic origin.

Transgene or foreign nucleic acid refers to a nucleic acid sequence encoding a protein of interest, that is inserted into the vector of the invention. At times, the transgene will be referred to simply as a “gene”. By “nucleic acid sequence encoding a protein of interest” is meant a sequence of a known gene of interest, including both the genomic sequence and the mRNA sequence, as well as sequences controlling the expression level of the mRNA or protein. This definition further comprises any modification of said sequence, including deletions, mutations, introduction of cellular transport-specific signals as are known in the art (e.g. a membrane-targeting signal, or signal peptide; ER or Golgi targeting signals, nuclear localization, etc.), or fragments of at least 20 base pairs (bp) thereof. Also included are sequences complementary to said nucleic acid sequences, i.e. antisense sequences, complementary sequences to inhibit expression (RNAi), cytokines and chemokines known to induce and fortify immune response.

Eliciting an immune response or inducing an immune response refers to activating either the humoral arm or the cellular arm of the immune system, or both. At times, this will also be referred to as “vaccination. Activation of the immune system may be assessed by any method known in the art, including production of antibodies and neutralizing antibodies; production or secretion of specific proteins such as interleukins, interferon, tumor necrosis factor (TNF), the induction of cytotoxic T lymphocytes (CTL) and any other indicators known in the art, and eliciting chemokines and cytokines known to attract lymphocytes and cytotoxic T cells to the site of infection.

Defective genome or replication-defective genome or defective virus all refer to a virus particle that is incapable of autonomous replication in a host cell. In particular, such a definition comprises the amplicon-6 and Tamplicon-7 vectors. Viral particles that have a defective genome will need a helper virus in order to replicate in a host cell.

Membrane associated refers to protein products that either have a transmembrane domain, or are capable of being modified in the cell such that they will be associated with the cell membrane. At times, these proteins will also be referred to as cell-surface associated proteins or proteins underlying the cell membrane. Among the known modifications, typical, but not exclusive examples include: acylation, amidation, GPI anchor formation, covalent attachment of a lipid or lipid derivative, myristoylation, pegylation, prenylation, palmitoylation, methylation, or any similar process.

Secreted protein—a protein designed for extracellular secretion.

Nucleic acid molecule or nucleic acid denotes a single-stranded or double-stranded polymer composed of DNA nucleotides, RNA nucleotides or a combination of both types and may include natural nucleotides, chemically modified nucleotides and synthetic nucleotides. This includes also oligomers and polymerase chain reaction (PCR) primers.

Amino acid sequence a sequence composed of any one of the 20 naturally appearing amino acids, and/or amino acids which have been chemically modified (see below), and/or synthetic amino acids.

Antibody—refers to antibodies of any of any class, including the classes IgG, IgM, IgD, IgA, and IgE antibodies. The definition includes polyclonal antibodies and monoclonal antibodies. This term refers to whole antibodies or fragments of antibodies comprising the antigen-binding domain of the anti-variant product antibodies, e.g. scFv, Fab, F(ab′)₂, other antibodies without the Fc portion, single chain antibodies, bispecific antibodies, diabodies, other fragments consisting of essentially only the variable, antigen-binding domain of the antibody, etc., which substantially retain the antigen-binding characteristics of the whole antibody from which they were derived. This definition also includes recombinant or synthetic antibodies and antibodies carrying toxic genes.

Treating a disease—refers to administering a therapeutic substance effective to prevent or ameliorate symptoms associated with a disease, to lessen the severity or cure the disease, or to prevent the disease from occurring. Treatment may also refer to slowing down the progression of the disease or the deterioration of the symptoms associated therewith, to enhancing the onset of the remission period, to slowing down the irreversible damage caused in the progressive chronic stage of the disease, to delaying the onset of said progressive stage, to improving survival rate or more rapid recovery, or a combination of two or more of the above.

The treatment regimen will depend on the type of disease to be treated and may be determined by various considerations known to those skilled in the art of medicine, e.g. the physicians.

Effective amount for purposes herein is determined by such considerations as may be known in the art. The amount must be effective to achieve the desired therapeutic effect as described above, i.e. eliciting an appropriate immune response. The amount depends, among other things, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the efficiency of expression of the desired protein, the efficiency of induction of an immune response against said protein, a variety of pharmacological parameters such as half life in the body, undesired side effects, if any, factors such as age and gender of the treated individual, etc.

Pharmaceutically acceptable carrier means any inert, non-toxic material, which does not react with the vectors of the invention. Thus, the carrier can be any of those conventionally used and is limited only by chemico-physical considerations, such as solubility and lack of reactivity with the compound, and by the route of administration. Examples of pharmaceutically acceptable carriers are detailed later on.

Vaccination vectors—a vector capable of inducing an immune response which is capable of eliminating the virus, or cells comprising and/or cancer antigens of the vaccination vector.

Cellular vaccination—use of autologous lymphoid cells such as dentritic cells containing and expressing the desired antigen.

Culture conditions means any conditions known in the art to enable the survival, gene expression and/or proliferation of mammalian cells. Such conditions may vary in accordance with the cell type in question. The conditions may comprise temperature, humidity, light intensity, providing of solutes, substrate or support, added cells, antibiotics, growth stimulating or inhibiting substances, energy source, metabolites, pH and the like. The growth conditions may also include procedures that need to be taken such as agitation of the cells or lack thereof and replenishment or replacement of any culture condition.

### SUMMARY OF THE INVENTION

The present invention concerns the use of amplicon-6 and Tamplicon-7 as vaccination vectors for efficient expression of selected genes in human lymphocytes. The main characteristics of the vectors of the invention are:

(i) The vectors contain large defective genomes of a total size corresponding typically to 135-150 kb potentially made of multiple reiterations of amplicon units and optionally carrying foreign DNA sequences of choice. For example, an amplicon may contain 10 reiterations of 15 kb repeat units. One could place several genes inside this unit, e.g. the gD and gDsec genes (see below), as well as Interferon chemokines and cytokines to enhance the immune response. Gene expression is efficient at least due to sequence reiterations.

(ii) The host range of the HHV-6 and HHV-7 vectors includes T cells, B cells, monocytes as well as dendritic cells. This is advantageous for vaccination inasmuch as lymphocytes express high levels of MHC class I molecules and induce strong immune response(s); the dendritic cells are efficient antigen presenting cells (APC).

(iii) The vectors are infectious entities, that may be used to bring transgene(s) into cells in vivo or ex vivo, which in turn may be followed by transplantation.

(iv) HHV-6A and HHV-7 are prevalent viruses which cause no known disease.

(v) The vectors are enable of expression of both cell surface-associated proteins as well as secreted proteins, thus ensuring a wide range of vaccination targets.

(vi) Efficient replication due to reiterations of cis acting replication signals.

(vii) Stability of gene expression (under HCMV promoter) for at least 7 days post superinfection. In fact, gene expression continues after 1-2 passaging.

(viii) No integration into the host genome, minimizing potential insertional mutagenesis.

(ix) Ability to target dividing and non-dividing cells.

In addition to the inherent safety factors of HHV-6A and HHV-7 described above, defective virus vectors are now described that do not damage the host cell, yet are capable of efficient expression of selected transgenes in lymphocytes and in dendritic cells known to have the capabilities of efficient MHC based antigen presentation.

The HHV-6 based vectors appear to be well suited for transfer of genes into lymphocytes which generally resist most common transfection methods, including calcium phosphate precipitation, electroporation, DEAE-dextran and lipofection.

According to one aspect of the invention, a lymphotropic vector is provided, optionally carrying one foreign gene or more, wherein administration of said vector to a mammal results in an immune response. Where the lymphotropic vector carries one foreign gene or more, the immune response may be against a product of at least one of the genes carried by said vector. In one embodiment, said foreign gene encodes a membrane-associated protein product or internal cellular gene products. In another embodiment, the foreign genes are soluble proteins, which may be secreted outside of the cell.

Thus, according to one aspect of the present invention, a lymphotropic vector is provided, comprising:

A DNA sequence derived from HHV-6 or HHV-7, said DNA sequence comprising an origin of replication, a cleavage and packaging signal and a promoter sequence which induces expression of at least one nucleic acid sequence product in a lymphocyte cell host;

wherein the administration of said vector to a mammal results in an immune response.

Optionally, said vector comprises at least one foreign nucleic acid sequence(s) capable of being expressed in said lymphocyte cell host.

In some embodiments, the vector is not capable of autonomous replication in a mammalian host cell, i.e. it comprises of a replication-defective genome. Optionally, said vectors may be replication-defective, thus enabling formation of concatamers, reiterated repeats of the gene of interest, and hence strong and efficient expression of the gene product in a host cell. In such cases it is preferred to add to a helper virus or cells comprising a helper virus.

In another embodiment, the replication defective lymphotropic vectors amplicon-6 or Tamplicon-7 infect the cells of the immune system, induce efficient gene expression, arouse immune response and then leave the scene upon lymphocyte divisions. The amplicon vectors may be propagated for elongated periods of time upon constant addition of a helper virus, such as HHV-6 or HHV-7 or by propagation in new cells carrying transfected amplicons. In a preferred embodiment, the helper virus is HHV-6A.

Examples of foreign nucleic acid sequences which may be inserted into the vector of the invention are GFP and B-gal markers, HSV-1 gD and gDsec, HIV-1 gp160 and REV, tumor antigens e.g., MUC1 protein for breast cancer immunotherapy, Prostate Specific Antigen (PSA), for Prostate cancer and Her-2 (neu) antigen for uterine serious papillary ovarian cancer immunotherpies. Additional genes added in amplicon forms or otherwise free form include adjuvant genes such as interleukines, cytokines and chemokines, designed to fortify the immune response.

MUC1 is an opportune tumor-associated antigen (TAA) for immune targeting since: (i) it is highly over expressed in malignant cells, (ii) the pattern of glycosylation is different in the protein expressed in malignant cells and in normal cells (iii) In malignant cells it is expressed all over the cell, whereas in normal cells it has normal apical distribution.

Employing the amplicon-6 MUC1 vector containing concatameric repeats of the gene, provides means to efficiently express the MUC1 protein in lymphocytes and dendritic cells as well as bring about secretion, of the protein outside the infected cells. Directing the expression to these cells, provides the means of targeting MUC1 into the class I pathways, to obtain efficient cancer immunotherapy.

According to another aspect of the invention, there is provided a method for eliciting an immune response in a mammal, said method comprising:

(a) providing a vector comprising a DNA sequence derived from HHV-6 or HHV-7, said DNA sequence comprising an origin of replication, a cleavage and packaging signal and a promoter sequence which induces expression of at least one nucleic acid sequence product in a lymphocyte cell host, optionally carrying a foreign nucleic acid sequence of interest;

(b) introducing said vector into the body of said mammal;

wherein said introduction results in an immune response in said mammal

According to yet another aspect of the invention, a method is provided for eliciting an immune response in a mammal, said method comprising:

(a) providing a vector comprising a foreign nucleic acid sequence;

(b) introducing said vector into lymphotropic cells; and

(c) introducing said lymphotropic cells into said mammal;

such that said introduction results in an immune response in said mammal.

Optionally, the immune response is against a protein product of nucleic acid sequence of interest in which case the foreign nucleic acid is nucleic acid sequence of interest.

The lymphotropic cells into which the vector is introduced may be any of dendritic cells, T cells and/or B cells. Preferably such cells are cells that are compatible for transplantation in said mammal, optionally being autologous cells derived from said mammal.

In a preferred embodiment of said method, said lymphotropic vector is derived from HHV-6 or HHV-7. Most preferably, said lymphotropic vector is either amplicon-6 or Tamplicon-7.

The lymphotropic vector in the methods described above may be according to any one of the embodiments described herein. The lymphotropic vector may be introduced as pure DNA, or as packaged amplicon-type defective virus or as infected cells containing vector DNA and foreign genes or along with a helper virus, or in other forms as will be described in more detail below.

The vectors of the invention are being used as safe means for inducing an efficient immune response in a mammal. Therefore, according to another aspect of the invention, there is provided a pharmaceutical composition comprising at least on of the vectors of the invention and a pharmaceutically acceptable carrier.

In yet another aspect of the invention, there is provided a kit comprising at least one of the vectors of the invention and a pharmaceutically acceptable carrier, and instructions for use. Optionally such kit also comprises a helper virus.

In the present invention, where a vector is introduced into a mammalian cell or used to prepare a pharmaceutical preparation or as part of a method of treatment, one may also add a helper virus to improve the replications of the vector. The helper may be provided in any form discussed in the present invention, including as bare DNA, as packaged DNA, as part of a virion and within cells comprising the helper. This may be especially useful in cases where the vector is replication defective.

A person skilled in the art of the invention would appreciate that the vector, virion and cell comprising the vector of the preset invention may be used as (or as a component of) a pharmaceutical preparation to illicit an immune response. In fact, in the present invention, where cells comprising a vector are prepared or used in order to administer them to a mammal, it is preferred that such cells would be compatible with the host so that they would not be rejected due to the host's immune response. It is thus preferred that the cells be such cells that are removed from the recipient in any manner known in the art.

In fact, cells comprising the vector of the invention may be used to produce any protein or transgene encoded by the vector, provided that the appropriate culture conditions are provided.

Finally according to yet another aspect, the present invention provides cells comprising the helper virus. It was shown that such cells are potentially better at enhancing the expression of genes carried by a vector than is the helper virus if provided to a culture without the additional cells (e.g. as naked DNA).

### BRIEF DESCRIPTION OF THE DRAWINGS

In order to understand the invention and to see how it may be carried out in practice, some embodiments will now be described, by way of non-limiting example only, with reference to the accompanying drawings, in which:

FIG. 1 is a scheme showing the structure of Amplicon-6 and Tamplicon-7, the insertion site of a foreign gene, and the generation of defective genomes with multiple repeats of the amplicon sequence.

FIGS. 2A-2C is a scheme showing the Tamplicon-7 vector system. FIG. 2A schematically shows pNF1182 and pNF1168 (pOrilyt, a construct that does not contain the packaging signals) which were used to construct Tamplicon-7 and Tamplicon-7.GFP. FIG. 2B depicts Tamplicon-7, containing the lytic replication origin (oriLyt) of HHV-7 and the packaging signals (pac) of HHV-7, and The Tamplicon-7.GFP, containing also the Green Fluorescent Protein (GFP), driven by the Human Cytomegalovirus promoter (HCMV). FIG. 2C shows a Southern blot analysis of nuclear (nuc) and cytoplasmic (cyto) DNA preparations and DNA from purified virions prepared from the medium (med.). M denotes a 1 kb DNA marker ladder.

FIG. 3 is a schematic diagram of the propagation of cell associated and cell free amplicon 6 vectors containing EGFP (Enhanced Green Fluorescent Protein).

FIGS. 4A-4F shows fluorescent microscope photographs of J-JHAN T cells that were transfected by electroporation with the vector Amp-6 EGFP (amplicon-6, containing EGFP). FIG. 4A shows Passage 0 (P0) , the electroporated culture viewed 7 days post-transfection (p.t.). FIG. 4B: P0, infected—cells were transfected and 48 hrs later superinfected with the helper virus HHV-6A (U1102). They were viewed 7 days p.t. FIG. 4C: Passage 1 (P1)—cultures which did not receive the HHV-6 helper virus were “passaged” by adding uninfected cells. FIG. 4D: P1 transfected/superinfected vectors were passaged to new, uninfected cells. Shown 1 week later. (E) P1 medium was filtered through 0.45 μm filters allowing passage of virus but preventing cell passage and producing “cell free vectors”. The filtered medium was used to infect new cells, which were inspected one week later. FIG. F is a scheme showing the structure of the amp-6-EGFP (pNF1194) plasmid.

FIGS. 5A-5B shows the results of two flow cytometry quantitations of GFP expression in J-JHAN T cells following transfection/superinfection. J-JHAN cells were first electroporated with amplicon-6-GFP vector, then superinfected with HHV-6A (U102) helper virus. Seven days post electroporation GFP expression was quantified by flow cytometry. Shown are duplicate cultures transfected and superinfected separately.

FIGS. 6A-6B shows passaging of amplicon-6-GFP vectors in T cells, infectious virus stocks with increased gene expression. Passaging of cell associated virus by adding uninfected J-JHAN cells (FIG. 6A); or by adding J-JHAN cells which received by electroporation the amp-6GFP vector (FIG. 6B). The resultant P1 virus secreted to the medium, can be employed to further infect J-JHAN cells or cells which received first amplicon-6 vector, generating P2 cell free (C.F.) virus stocks. The fraction of cells infected and the MFI are estimated by flow cytometry.

FIG. 7 depicts the production of virus stocks with increased infection capacity, showing passaging of amplicon-6-GFP vectors in T cells by infecting J-JHAN cells, or J-JHAN cells which received by electrophoresis the amplicon-6 GFP vector. This generated virus stocks with increased gene expression.

FIG. 8 is a picture of the Immunofluorescence observed in mock infected KM-H2 B cells, showing that there is no detectable background fluorescence.

FIGS. 9A-9B shows a picture of the Immunofluorescence observed in Amplicon-6-GFP infections of J-JHAN (FIG. 9A) cells and KM-H2 B cells (FIG. 9B).

FIGS. 10A-10B shows flow cytometry of T cells and B cells infected with amplicon-6 GFP vector.

FIGS. 11A-11C graphically show dose dependence of amplicon-6-GFP vector infection of B cells. KMH2 B cells were infected with different doses of Amplicon-6-GFP. Viral infection was monitored by FACS analyses. The doses were 10 μL virus (FIG. 11A), 20 μL virus (FIG. 11B), and 40 μL virus (FIG. 11C).

FIGS. 12A-12D show 4 samples of immature dendritic cells preparation as viewed in Zeiss Microscope, 5 days after preparation from adherent cells treated with GMCSF and IL-4.

FIGS. 13A-13D show 4 samples of mature dendritic cell preparation viewed in a Zeiss Microscope. At day 5 after treatment with GMCSF and IL-4 the cells were treated for 48 hrs with PGE2, and IL-4TNF.

FIGS. 14A-F depict flow cytometry analyses of dendritic cell preparations. The immature (FIGS. 14A, 14C and 14E) and mature (FIGS. 14B, 14D and 14F) dendritic cells were analyzed for expression of CD1A, CD83 and CD86, respectively.

FIGS. 15A-15D are fluorescent microscope photographs of dendritic cells infected with Amp6-GFP vector prepared as cell free virus from the medium of infected cells. Two samples are shown, one sample in FIGS. 15A and 15B and the other in FIGS. 15C and 15D. FIGS. 15A and 15C show phase contrast exposure of the samples combined with fluorescence and FIGS. 15B and 15D show the fluorescence exposure of the same cultures, respectively.

FIGS. 16A-16B shows the structure of amplicon-6 containing an intact gD gene driven by the HCMV promoter - Amp6-gD (FIG. 16A), and amplicon-6 containing the gD gene with a 201 bp deletion of the transmembrane signal—Amp6-gDsec (FIG. 16B).

FIG. 17 shows the expression of Amp6-gD mRNA in J-JHAN cells with and without super infection with the HHV-6 helper virus. Lanes 1 and 2—Expression of gD MRNA from cells electroporated with Amp6-gD at 24 and 48 hrs post transfection, assessed by reverse transcriptase (RT). Lane 3—Vero cells infected with HSV-1. Lane 4—plasmid DNA of the Amp6-gD vector. Lanes 5 and 6 are negative controls identical to lanes 1 and 2 but without the reverse transcriptase (RT) enzyme. Lane 7—DNA marker. Lane 8—same as lane 3, without RT.

FIG. 18 is a Western blot analysis of the expression of Amp6-gD and gdsec in J-JHAN T cells. The blot was probed with anti-gD monoclonal antibodies (mAbs). Lane 1—proteins of HSV-1 infected Vero cells (Monkey kidney cells used for HSV propagation). Lane 2—marker. Lane 3—mock transfection. Lane 4—J-JHAN cells transfected with Amp6-gD, 7 days p.t. (post-transfection). Lane 5—J-JHAN cells transfected with Amp6-gDsec, 7 days p.t.

FIG. 19 A Western blot analysis of the expression of Amp6-gD in J-JHAN T cells, which were transfected with Amp6-gD with and without HHV-6 helper virus. The blot was probed with anti-gD mAbs. Lane 1—Vero cells infected with HSV-1. Lane 2—protein size marker. Lane 3—J-JHAN cells infected with helper virus, but not with Amp6-gD. Lane 4—J-JHAN cells infected with Amp6-gD, but not with helper virus. Lane 5—J-JHAN cells infected with both helper virus and Amp6-gD. Lanes 6 and 7—filtered medium of passage 0 cells without (lane 6) and with (lane 7) helper virus, was used to infect new cells. Seven days later, the proteins were analyzed. Lanes 8 and 9—Passage 1 of the cell-associated Amp6-gD without (lane 8) and with (lane 9) helper virus.

FIG. 20 A Western blot of J-JHAN T cells, which were transfected with Amp6-gDsec with and without HHV-6 helper virus. The blot was probed with anti-gD mAbs. Lane 1—Vero cells infected with HSV-1. Lane 2-Protein size marker. Lane 3—J-JHAN cells infected with Amp6-gD, but not with helper virus. Lane 4—J-JHAN cells infected with both helper virus and Amp6-gD. Lanes 5—the medium of passage 0 Amp6-gDsec vector with helper virus was filtered, concentrated and used to infect new cells, generating cell passage 1. At the end of the infection proteins were prepared and analyzed in the Western blot probed with anti-gd antibodies. Lanes 6 and 7—Passage 1 of Amp6-gDsec propagated from the vectors with (lane 6) and without (lane 7) helper virus. Lane 8—Passage 2 of the Amp6-gDsec vector/superinfected cells.

FIG. 21 A Western blot of trichloroacetic acid (TCA) precipitation of gDsec or gD from the medium of J-JHAN T cells, which were transfected with Amp6-gDsec or Amp6-gD, with and without HHV-6 helper virus. The blot was probed with anti-gd mAbs. Lane 1—Vero cells infected with HSV-1. Lane 2—protein size marker. Lane 3—Passage 0 (P0) of J-JHAN cells transfected with Amp6-gDsec, 48 hrs post-transfection (p.t.), without helper virus. Lanes 4 and 5—TCA precipitated medium of the PO gdsec culture, 48 hrs (lane 4) or 7 days (lane 5) p.t., without helper virus. Lane 6—TCA precipitated medium of the P0 gDsec culture, 7 days post-transfection, with helper virus. Lanes 7-10—transfections with Amp6-gD including electroporated cells (lane 7) and TCA precipitated medium of the PO electroporated Amp6-gD, 2 and 7 days p.t. without helper virus (lanes 8 and 9) and with helper virus (lane10). The TCA precipitated proteins appeared to be smaller than the non-TCA precipitated proteins, indicating breakage.

FIGS. 22A-22F shows confocal microscope images of J-JHAN cells infected with both HHV-6 helper virus and Amp6-gD. The cells were stained with the H170 anti-HSV-gD antibody. Each figure is composed of: upper left- fluorescent photo, upper right-differential interactions contrast (Nomarsky) photo, and the lower left-superposition of the fluorescent and Nomarsky photo. (FIG. 22A) HHV6A (U1102) infected J-JHAN cells. FIGS. 22B-22F) Representative images of J-JHAN cells transfected with Amp6-gD and superinfected with HHV6A (U1102). In FIGS. 22B-22D the scale bar depicts 10 μm. In FIG. 22A the scale bar depicts 20 μM.

FIGS. 23A-23E Confocal microscope images of J-JHAN cells infected with both HHV-6 helper virus and with Amp6-gDsec. The cells were stained with the H170 anti-HSV-gD antibody. A-E—Representative images of J-JHAN cells that are transfected with Amp6-gDsec, and superinfected with HHV6A (U1102). Each part of the figure (e.g. A) is composed of: upper left—fluorescent photo, upper right—differential interactions contrat (Nomarsky) photo, and lower left-superposition of the fluorescent and Nomarsky photos. In FIGS. 23A-23B the scale bar depicts 20 μm. In FIGS. 23C-23E the scale bar depicts 10 μm.

FIGS. 24A-24D show dot plots of flow cytometry of amplicon-6-gD transfected J-JHAN cells with and without superinfecting helper virus. FIG. 24A: Cultures of uninfected cells, FIG. 24B: cultures infected with helper virus only.

FIG. 24C: Cells electroporated with amplicon-6-gD. FIG. 24D: Cells receiving both the amplicn-6-gD and the helper HHV-6A (U1102). FIG. 24E: depicts the mean fluorescence intensity (MFI) of the above different cultures.

FIGS. 25A-25D depicts flow cytometry of amplicon 6 vector infection to quantitate efficiency of gene expression in T cells. FIG. 25A: Cultures of uninfected J-JHAN cells. Less than 1% of the cells show background fluorescence. FIG. 25B: cells were electroporated with amplicon-6 gD. 16% of the cells show fluorescence. FIG. 25C: cultures infected with helper virus only. <5% of the cells show fluorescence. FIG. 25D: Cells receiving both the amplicon-6 gD and the helper virus. 80% of the cells show fluorescence.

FIG. 26 is a scheme showing the level of gD expression by the flow cytometry. Shown are the mean fluorescence intensities (MFI) in the different J-JHAN cultures of FIGS. 25A-25D probed in the flow cytometry, employing the gD antibody.

FIG. 27 is a schematic representation of Amplicon-6 vectors carrying the HIV-1 gp160 gene and both gp160 and REV genes.

FIG. 28 is a Western blot analysis of amplicon-6-gp160 expression in 293 cells. The blot was probed using anti-gp120 1A8 mAbs. Lane 1—control, mock transfected cells. Lane 2—cells transfected with Amp6-gp160. Lanes 3 and 4-cells transfected with mixtures of both amplicon-6-gp160 and amplicon-6-REV clone a (lane 3) and clone b (lane 4). Lanes 5 and 6—cells transfected with clone 9 (lane 5) or clone 15 (lane 6) of Amp6-gp160-REV. Lane 7—purified gp120 as positive control.

FIG. 29 shows a Western blot analysis of Amp6-gp 160-REV expression in J-JHAN cells with and without HHV-6 helper virus. The blot was probed using the anti-gp120 1A8 mAb. Lane 1—pure gp160 as positive control. Lane 2—protein size marker. Lane 3—untransfected cells. Lane 4—cells infected with helper virus but no amplicon vector. Lanes 5 and 6—P0 cells transfected with amplicon-6-gp160-REV without (lane 5) and with (lane 6) helper virus. Lanes 7 and 8—filtered medium of P0 Amp6-gp160-REV transfection without (lane 7) and with (lane 8) helper virus. The filtrate was passaged to new cells which were tested 7 days later. Lanes 9 and 10—passage 1 of cell-associated Amp6-gp160-REV without (lane 9) and with (lane 10) helper virus.

FIG. 30 shows a Western analysis of propagated Amp6-gp160-REV in J-JHAN cells. The blot was probed using anti-gp120 1A8 mAb. Lane 1—untransfected cells. Lane 2—cells infected with helper virus but without the amplicon vector. Lanes 3 and 4—P0 cells transfected with Amp6-gp160-REV without (lane 3) or with (lane 4) helper virus. Lane 5—passage 1 of cell associated Amp6-gp160-REV with helper virus. Lane 6—passage 2 of cell associated Amp6-gp160-REV with helper virus. Lane 7—filtered P1 Amp6-gp160-REV with helper virus was passaged to new cells, generating P2 infection cultures, which were assayed 7 days later. Lane 8—the medium of P2 cells was filtered and passaged to new cells which were assayed 7 days later.

FIGS. 31A-31B shows a confocal microscope analyses of J-JHAN cells transfected with Amp6-gp160-REV and superinfected with the U1102 helper virus. Cells were stained using the anti-gp120 1A8 mAb. Each part of the figure is composed of: upper left—fluorescent image, upper right—Nomarsky imaging, lower left—superposition of the two images. (FIG. 31A) Cells infected only with helper virus. (FIG. 31B) Cells transfected with Amp6-gp160-REV and superinfected with the U1102 helper virus.

FIGS. 32A-32B shows confocal microscope analyses of J-JHAN cells transfected with Amp6-gp160-REV and superinfected with the U1102 helper virus. Cells were stained with the CG10 anti-gp120-CD4 complex mAb. Each part of the figure is composed of: upper left—fluorescent image, upper right—Nomarsky imaging, lower left—superposition of the two images. (FIG. 32A) Cells infected only with helper virus. (FIG. 32B) Cells transfected with Amp6-gp160-REV and superinfected with the U1102 helper virus.

FIG. 33 shows Western blot analysis of Amplicon-6-MUC1 expression in 293T cells. eIF2α antibody was employed to confirm equal protein loading, and the blot was probed with the anti-MUC-1 monoclonal antibody. Each lane, not including the “ladder”, shows the following: uninfected 293T cells (lane 1), 293T cells transfected with amplicon-6-GFP control (lane 2) and cells transfected with the amplicon 6 MUCI vector (lane 3).

FIG. 34 shows Western blot analysis of Amplicon-6-MUC1 expression in J-JHAN cells. eIF2α antibody was employed to confirm equal protein loading, and the blot was probed with the anti-MUC-1 monoclonal antibody. Each lane shows the following: Ladder for size comparison (lane 1), J-JHAN mock infected cells (lane 2), cells infected with HHV-6 U1102 (lane 3), cells infected with amplicon-6-MUC-1 (lane 4) and cells infected with amplicon 6 MUC1 and superinfected with HHV-6U1102 helper virus at passage 0 (lane 5) and after cell associated passage 1.

FIG. 35 shows TCA precipitation of secreted MUC-1 protein in the medium of J-JHAN cells infected with amplicon-6-MUC1 with and without HHV-6 helper virus. Each lane shows the following: Ladder for size comparison (lane 1), cells infected with amplicon-6-MUC1 and helper HHV-6AU1102 without TCA precipitation (lane 2), TCA precipitation of the medium of mock infected J-JHAN cells (lane 3), cells infected with HHV-6A (U1102; lane 4), cells infected with amplicon6-MUC-1 (lane 5) and cells infected with amplicon6-MUC-1 and the helper HHV-6A (U1102; lane 6).

FIGS. 36A-36D depicts confocal microscope analyses of amplicon-6-MUC1 infected J-JHAN cells. J-JHAN cells were infected and viewed in a confocal microscope, employing the MUC1 antibody. In each figure upper left-fluorescent photograph, upper right—differential interactions contrast (Nomarsky) photograph, lower left—superposition of the fluorescent and Nomarsky photographs. FIGS. 36A-36C show cells infected by amplicon-6-MUC and helper HHV-6 (U1102). FIG. 36D. show cells infected by the helper HIV-6 (U1102) only.

FIGS. 37A-37D depicts confocal microscope analyses of amplicon-6-MUC1 infected J-JHAN cells after perforation with Triton X100. J-JHAN cells were perforated with Triton X100 and then exposed to high serum suspended in order to block non-specific antibody reaction. The confocal microscope staining employed the MUC1 antibody. In each figure upper left-fluorescent photograph, upper right-differential interactions contrast (Nomarsky) photograph, lower left-superposition of the fluorescent and Nomarsky photographs. FIGS. 37A-37C show cells infected by amplicon 6-MUC and helper HHV-6 (U1102). FIG. 37D. show cells infected by the helper HHV-6 (U1102) only.

FIGS. 38A-38D show MUC1 expression in T cells infected with amplicon-6MUC1 vector with and without HHV-6 superinfection, as measured by FACS. FIG. 38A: Mock infection. FIG. 38B: helper HHV-6A (U1102) infection. FIG. 38C: Transfection with amplicon-6-MUC1 vector. FIG. 38D: Transfection with amp-6-MUC1 vector and superinfection with helper virus. Shown are % infected cells and MFI levels.

### DETAILED DESCRIPTION OF THE INVENTION

The composite amplicon vectors of the invention may comprise two components: (i) defective genomes with multiple reiterations of amplicon units, each containing the DNA replication origin and packaging signals, as well as the selected transgene(s). (ii) an adequate helper virus which provides the DNA replication and packaging functions and the structural particle. In the presence of the helper virus the amplicons replicate by the rolling circle mechanism, producing large concatamers of the input amplicons with the signals pac-1 and pac-2, located at the junctions between repeats. The concatamers are cleaved 29-35 bp away from the pac-1 signal and 40-45 bp away from the pac-2 signals, located at approximately “headfull” or full length genomes, resulting in defective genomes of overall size 135-150 kb made of multiple reiterations of amplicon units (Romi et al., 1999). The defective viruses follow their nondefective helper viruses in their cell tropism and ability to infect dividing as well as non-dividing cells. HHV-6 was shown to infect mature T lymphocytes, lymph nodes, macrophages and monocytes, dendritic cells, kidney tubule endothelial cells as well as CNS tissues.

The defective amplicon virus vectors of the invention are capable of efficient expression of selected transgenes in lymphocytes and dendritic cells known to have the capabilities of efficient MHC based antigen presentation. As described below the system was assayed employing the GPF marker gene, the gD and gDsec genes to inhibit facial and genital herpes infections, the HIV glycoprotein gp160, towards development of an AIDS vaccine and the tumor antigen MUC1 to create an anti-cancer vaccine. The amplicon-6 vectors are expressed most efficiently in T cells, B cells, dentritic cells (see below). Immunization experiments using purified defective virus DNAs and virus vectors with or without helper viruses are currently ongoing in human peripheral blood systems and in animals. Vaccination is being tested for both humoral and cellular immunization. Vaccination against different herpes viruses and other viruses may be done be inserting the relevant genes of interest into the amplicon vectors of the invention, and introducing them to the immune system by means which will be described below. Vaccination efficiencies can be improved significantly with the amplicon-6 vector relative to existing genetic vaccination systems, due to the sequence reiterations of the vectors of the invention, which give rise to a high level of expression of the DNA sequence(s) of interest.

The transport of the gD gene into lymphocytes out of the virus grown in epithelial and mucosal cells is expected to significantly increase efficiency, inasmuch as the natural HSV contains functions that are known to escape and evade the immune system. Furthermore expression of multiple copies per cell is most efficient and results in overproduction of the selected DNA sequences.

Finally, an additional extension for the potential use of amplicon-6 vectors for efficient antigen presentation in lymphocytes includes cancer vaccination employing proteins which have abnormally high expression in malignant cells and tissues e.g., the MUC1 protein in breast cancer, and the Prostate Specific Antigen (PSA) for prostate cancers. Greater efficiency in vaccine production is predicted.

Examples of the lymphotropic vaccination vectors of the invention are:

(a) amplicon-6

(b) HHV-6 helper, capable of binding to the CD46 receptor;

(c) a mutant of HHV-6;

(d) segments of the vectors of above (b) and (c) which can provide amplicon helper functions;

(e) HHV-6 helper BAC clones, devoid of packaging signals, but capable of providing helper virus functions

(f) helper cell lines derived from (d) and (e)

(g) any combination of the agents under (a) to (f).

Similar vectors employing Tamplicon-7, HHV-7 helper virus, fragments of helper virus DNAs, BAC HHV-7 and helper HHV-7 cell lines.

In addition to the DNA sequences derived from HHV-6 or HHV-7, the vectors of the invention comprise an origin of DNA replication, cleavage and packaging signals, a promoter sequence capable of inducing expression of downstream nucleic acid sequences in host blood cells. The vectors may optionally comprise also foreign nucleic acid sequences downstream to an expression control of said promoter sequence.

For therapeutic use, said lymphotropic vector may be incorporated into a delivery vehicle. A large number of vehicles are available for the delivery of genetic material into cells, delivery vehicle which are viral-derived particles are generally preferred in view of the specificity of such particles to certain cells which facilitate the targeting of the genetic material to such cells. Seeing that the lymphotropic vector of the invention is derived from HIIV-6 or HHV-7, the preferred viral particle for use as a delivery vehicle is derived from these two respective viruses. There is some evidence that HHV-7 may activate HHV-6 replication (Katsafanes et al., 1996), and accordingly, it is also possible in accordance with the invention to use an HHV-7 particle as a delivery vehicle for an HHV-6 derived lymphatic vector.

HHV-6 or HHV-7 particles are known to have an affinity to specific cell types. The HHV-7, binds to the CD4 receptor and accordingly the particle derived from the HHV-7 is useful for the delivery of said lymphotropic vector to CD4⁺ cells. The HHV-6 particles bind CD46 receptor and have an affinity to a variety of cells and mainly to both CD4⁺ and CD8⁺ cells, as well as to some other blood cells, e.g. EBV infected, as well as EBV negative B-cells, and may thus be useful for the targeting of said lymphotropic vector to such cells, as well as to dendritic cells which are the most efficient antigen presenting cells.

The preferred delivery vehicle in accordance with the present invention, is selected from:

(a) an HHV-6 or HHV-7 particle;

(b) a mutant HHV-6 or mutant HHV-7 particle capable of infecting lymphatic cells and delivering its content of DNA to such cells;

(c) a chemically modified particle of (a) or (b) essentially retaining the ability to infect lymphatic cells; and

(d) any combination of (a), (b) or (c).

Several kinds of vectors are provided by the present invention: a helper virus vector which is capable of autonomous replication (hereinafter: “ARV” (autonomously replicating vector)); a vector which is not capable of self replication (hereinafter: “amplicon”). While an ARV may be administered by itself, an amplicon is administered together with a helper virus which provides the transactivation factors for replication of the amplicon. The choice of the helper virus may typically be based on the nature of the amplicon: in case of an amplicon derived from HHV-6, a self-replicating HHV-6 will typically be used, preferably HHV-6A. In the case of a Tamplicon derived from HHV-7, a self-replicating HHV-7 will typically be used. As pointed out above, a self-replicating HHV-7 may be used as a helper virus for an HHV-6 derived amplicon. Alternatively, HHV-6A may be used as a helper virus for an HHV-7 derived Tamplicon. The composite amplicon-6A vectors are non-replicating and after 2-3 passages they disappear in vivo.

As already pointed out above, HHV-6A and HHV-7 have no known independent pathology and therefore their use as helper viruses is generally preferred where possible over the use of HHV-6B. Use of HHV-7 is limited primarily to CD4⁺ cells and accordingly use of HHV-6A is at times preferred. In case use is made of the HHV-6, measures may be further taken to neutralize this virus. A mutant HHV-6A may be used, the expression of which may be controlled by changes in various factors such as, a change in temperature (i.e. a temperature sensitive mutant). Alternatively, a deletion mutant in potential biohazard functions can be used.

The helper virus functions can be provided by superinfecting virus, or by co-transfection with large DNA clones, or by first cloning the entire genome lacking packaging signals in large bac vectors, then placing all genes in a cell line on top of which the defective genomes with multiple copies of the amplicons may be placed and used for production of pure defective vectors. Additionally, integration may be obtained by introducing the neogene and also adenovirus associated virus (AAV) into the helper or defective amplicon vectors. Potentially the pure vectors can be also be used for vaccination.

Mutant viruses may be obtained by standard methods. An example of a mutant is such which is unable to replicate by itself in a host cell. Another type of mutant may, for example, be such which has a higher affinity to binding to the CD4 receptor than the native strain.

A particle of the virus may be obtained by various standard methods which are known in the art. Various polypeptides, are obtainable either by chemical methods or by methods of genetic engineering, namely, by cloning and expressing a gene coding for the polypeptide. Such a polypeptide is typically a portion of the virion which determines the binding affinity to the CD4 receptors in the HHV-7 or CD46 receptors in HHV-6 vectors. Polypeptides produced by means of genetic engineering can sometimes be obtained as fusion proteins of the desired polypeptide with another protein or peptide component. Such fusion proteins may also be useful at times as said CD4-ligand, or CD46 ligands.

Derivatives may be obtained by various standard chemical or biochemical methods, or by methods of genetic engineering, such methods being generally known per se.

The specific regimen for vaccination can be determined for each antigen by routine methods known to those skilled in the art. In each case, the vaccination regimen should ensure an effective amount of antigen will be presented to the immune system of the subject. For some antigens, a high in vivo level of the vaccination agent (i.e. the lymphotropic vaccination vector) in the blood may be desirable. In such cases, the use of an ARV or of a helper virus may be preferable.

In other cases, it may be desirable to stop the expression of the lymphotropic vector at a desired point of time. In such case, it is preferable to use a helper virus or even a lymphotropic vector with a carrier vehicle, but without a helper virus.

As mentioned above, the invention provides pharmaceutical compositions comprising any one of the vectors of the inventions, along with a pharmaceutically acceptable carrier. As described above, a pharmaceutically acceptable carrier is any inert, non-toxic material, which does not react with the vectors of the invention. The carriers may also refer to substances added to pharmaceutical compositions to give a form or consistency to the composition when given in a specific form, e.g. in a form suitable for injection, spray, aromatic powder etc. The carriers may also be substances for providing the composition with stability (e.g. preservatives).

The choice of carrier will be determined in part by the particular vector, as well as by the particular method used to administer the composition. Accordingly, there is a wide variety of suitable formulations of the pharmaceutical composition of the present invention. A preferred formulation is that suitable for parenteral administration, for example subcutaneous, intravenous, intraperitoneal or intramuscular, either systemically or locally. The requirements for effective pharmaceutical carriers for injectable compositions are well known to those of ordinary skill in the art. See, for example, Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986). It may also be administered by intravenous infusion.

As an example, for the preparation of a pharmaceutical composition suitable for viral vector or infected cell administration, e.g. intravenously by iv drip or infusion.

Carriers suitable for injectable formulations of the compositions of the invention may include, without being limited thereto, Interleukin solutions, chemokines and cytokines, vegetable oils, dimethylacetamide, dimethylformamide, ethyl lactate, ethyl carbonate, isopropyl myristate, polyols (glycerol, propylene glycol, liquid polyethylene glycol, and the like). For intravenous injections, water-soluble versions of the therapeutic agent may be administered by the drip method, whereby a pharmaceutical formulation containing a vector and a pharmaceutically acceptable carrier is infused. Specific pharmaceutically acceptable carriers may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

As should be appreciated, the pharmaceutical composition may be in the form of a medical formulation kit, together with at least one type of medical carrier or diluent.

Materials and Methods

Antibodies

Primary Antibodies:

H-170—A mouse anti-gd IgG, recognizes linear epitope at the N-terminal of HSV-1 and HSV-2 gD (gift of Dr. Lenore Pereira, Department of Stomatology, school of Dentistry, University of California, San-Francisco, USA).

1A8—A mouse anti-gp120 IgG, recognizes linear epitope at the N-terminal of HIV-1 gp120. (gift of Prof. Jonathan Gershoni, Tel Aviv University).

cg10—A mouse anti-gp120-CD4 complex IgG antibody (gift of Prof. Jonathan Gershoni, Tel Aviv University).

MUC1 antibody H-23—a mouse anti-human MUC1 monoclonal antibody, which recognizes tandem repeats of the MUC1 glycoprotein (obtained from Prof. Daniel Vreshner, Tel Aviv University).

Secondary Antibodies:

Goat Anti-Mouse IgG antibody- Cy™3-conjugated conjugated affinipure Goat anti-mouse IgG F(ab′)₂, bought from Jackson immunoResearch laboratories. (Code no. 115-166-072, Lot: 40709).

Goat Anti-Mouse IgG antibody- FITC-conjugated affinipure Goat anti-mouse IgG (H+L), bought from Jackson ImmunoResearch laboratories. (Code no. 115-095-062).

Goat anti-mouse IgG antibody—peroxidase-conjugated affinipure Goat anti-mouse IgG (H+L), bought from Jackson ImmunoResearch laboratories. (Code no. 115-035-146, Lot no. 51633).

Secondary Antibody PE; Donkey anti mouse IgG(H+L)—R-phycoerythrin conjugated affinipure F(ab)₂, bought from Johnson Immunoresearch Laboratories.

Cell Cultures and Viruses

Cell Cultures

Two human CD4+ T-lymphocytes cell lines were used: J-JHAN cells—derived from Jurkat T cells, and Sup-T1 cells—derived from a non-Hodgkin's T-cell lymphoma patient (ATCC CRL-1942). Both cell lines were propagated in RPMI 1640 medium, supplemented with 10% heat-inactivated fetal calf serum (FCS), 2 mM L-glutamine (Biological Industries), and 50 μl from 50 mg/ml of gentamicin stock. The KMH2 B cell line is an EBV negative line derived from a human Hodgkins lymphoma patent. These cell lines grow in suspension. 293T cells are propagated in DMEM medium, supplemented with 10% (FCS), 2 mM L-glutamine (Biological Industries), and penicillin (20 U/ml), streptomycin (20 μg/ml), nystatin (2.5 U/ml): (PEN-STREP-NISTATIN, Biological industries). All cell cultures were incubated at 37° C. in a humidified 5% CO₂ incubator. Dendritic Cells (DC) Peripheral blood mononunonuclear cells (PBMC) are prepared from approximately 70 ml of blood using lymphoprep (Bet Haemek, Israel). After removal of red blood cells the PBMC non-adherent cells are removed and saved for subsequent T-cell isolation. The adherent cells are cultured in RPMI supplemented with granulocyte macrophage colony stimulation factor (GMCSF) (0.1 μg/ml) and interleukin-4 (IL-4) (0.05 μg/ml). The cells are incubated for 5-6 days, changing the medium every 2 days to obtain immature dendritic cells. DC maturation is accomplished by the addition of Tumor Necrosis Factor (TNF). As will be further shown the immature and mature dendritic cells express the CD1A, CD83 and CD86 dendritic as expected.

Viruses and in vitro Infection

The HHV-6A strain U1102 was obtained from Dr. Robert Honess and propagated in J-JHAN or in the cell line SUP-T1 cell lines. The viruses were propagated by cocultivation of infected cells with fresh uninfected cells (1:1 ratio). Uninfected cells were incubated with infected cells for 2 hr at 37° C. in a humidified 5% CO₂ incubator, in a concentrated aliquots of volume <1 ml for absorption of the virus. After the absorption, the infected cells were diluted into RPMI 1640 medium containing 10% fetal calf serum. Infection was assessed by the appearance of a cytopathic effect characterized by marked enlargement of infected cells and formation of syncytia.

Transfection and Superinfection

J-JHAN cells (400 μL at concentrations of 10⁷ cells/ml) in RPMI-1640 medium were electroporated with 50 μg purified plasmid DNA in 4 mm gap disposable cuvettes (BIX P/N 640) by one pulse at 250 V, 24 msec using the Electro cell manipulator ECM 395. The electroporated cells were incubated for 10 min on ice and then transferred to 5 ml RPMI 1640 with 10% fetal calf serum and 50 μg/ml gentamicin at a final concentration of 4×10⁶ cells/ml. At 24 h-48 h after electroporation the cells were superinfected with equal number of HHV-6 (U1102) fully infected cells. The cultures were harvested for further passaging and protein extraction.

Plasmid Construction

All the amplicon-6 final transgenes used have the human cytomegalovirus (HCMV) promoter and the SV40 polyadenylation signal and were prepared in E. coli DH10B or the E. coli K12 GM2163 (DAM⁻/DCM⁻) bacteria, using the Nucleobond AX plasmid maxi prep kit of Macherey-Nagel, Germany.

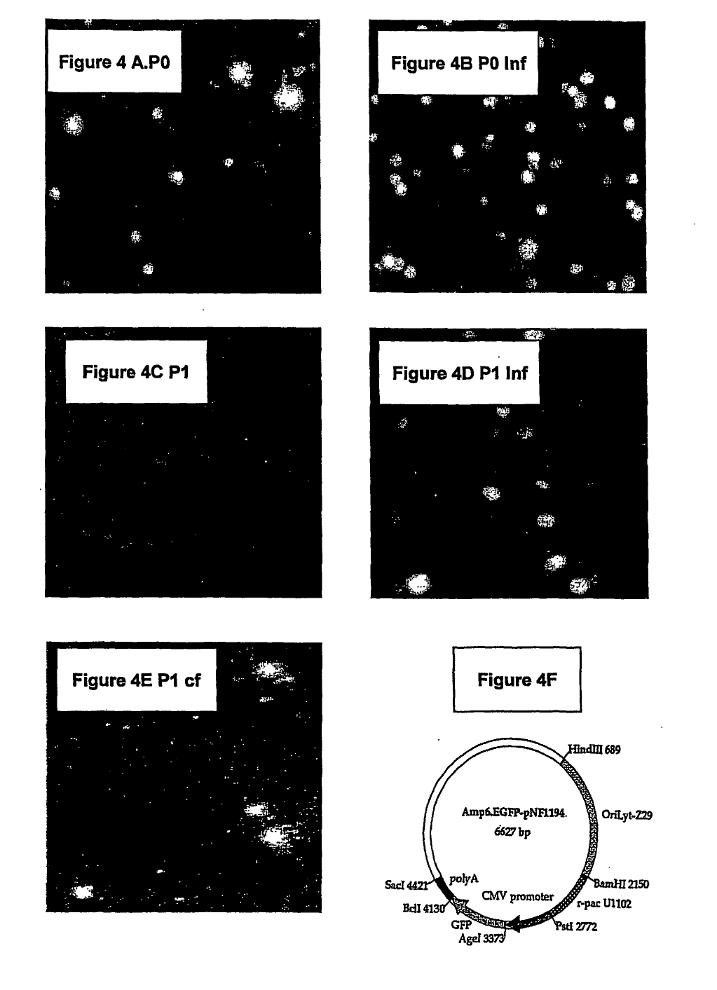

To derive the amplion-6-GFP plasmid vector we utilized the GFP plasmid (pEGFP-C3, from Clontech), which contains a multi cloning site linker at the C-terminus of the GFP gene, designed for protein fusions. The linker was removed and the coding region was cloned in pBluescript II SK (Stratagene) (pNF1193). A fragment that contained both the recombinant packaging (r-pac) signal of HHV-6A and the lytic origin of DNA replication (r-pac/oriLyt fragment) was cloned in pNF1193 and the final construct was designated Amplicon-6.EGFP (pNF1194) (FIG. 4F).

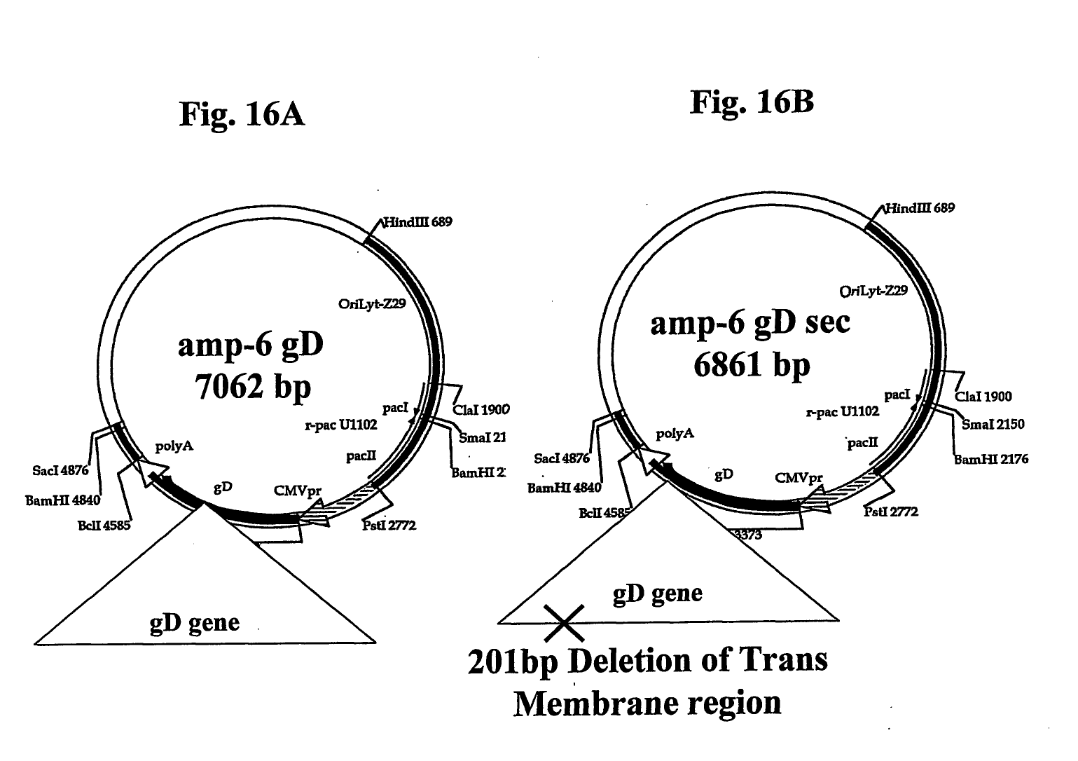

Amplicon 6-gD (pNF 1215) (FIG. 16A)—and the Amplicon-6-gDsec (pNF1219)(FIG. 16B) contain the gD sequences of HSV-1 (F). The gD gene was derived by PCR of the BamHI-J fragment of HSV-1 (F) (clone pNF 417). Two PCR primers containing oligonucleotide tails with the AgeI and BclI restriction enzyme sites were used: sense including the AgeI site: 5′- CAG CTT CAC G acc ggt AG GTC TCT TTT GTG TGG TGC -3′ and anti sense, including the BclI site: 5′-GAT ACT AGC C tga tca GG GGT ATC TAG TAA ACA AGG -3′. These sites match the AgeI and BclI (shown in small letters) bounding the CMV promoter and the SV40 poly A signal of the amplicon-6-GFP (pNF 1194) described above. The amplicon-6-gD construct (pNF1215) was produced in E. coli K12 GM2163 (DAM⁻/DCM⁻) competent bacteria. The gD fragment, digested with AgeI and BclI restriction enzymes was ligated into the parallel sites of the pNF1194 fragment substituting GFP gene. The resultant colonies were screened by PCR picking. A number of the positive colonies were sequenced and compared with the original sequence, using NCBI/Blast. The matching plasmid amplicon-6-gD (pNF1215) contains the intact gD gene. To construct a secreted form of the gD gene, the transmembrane region (TMR) of the gD gene was deleted by PCR. The Amp6-gDsec contains the first 327 amino acids (aa) of the gD 1 gene and lacks 67 aa at the carboxy terminus, which include the transmembrane region (TMR). The gDsec antisense including the BclI site (small letters) and stop codon (underlined) was: 5′-ACT AGC C tga tca CT AGG CGT CCT GGA TCG ACG G -3′. The gDsec fragment was digested with the AgeI and BclI restriction enzymes and ligated into the parallel sites on the pNF1194 vector resulting in the amplicon-6-gDsec (pNF 1219) (FIG. 16B). Overall the gD constructs have the HCMV promoter and the SV40 polyA.

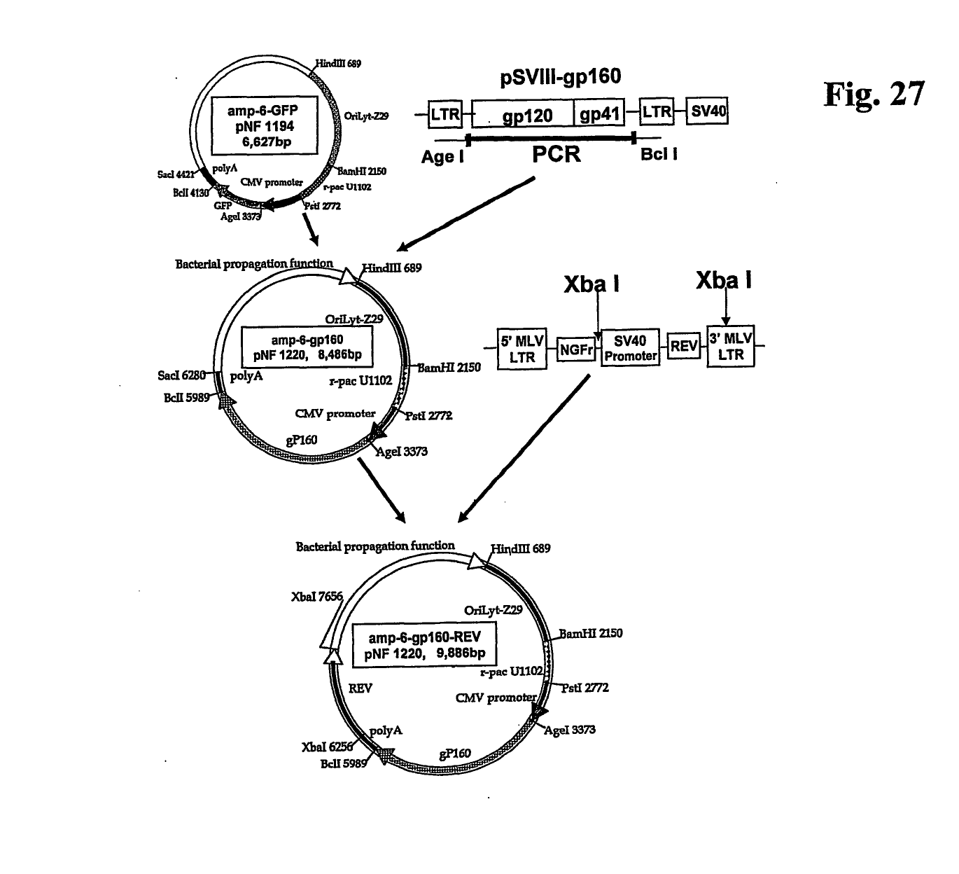

Amp6-gp160 (pNF1220) (FIG. 27) contains the gp160 gene of HIV subtype B from pSVIIIgp160—clone 92HT593.1, gene bank accession no. U08444, received from the NIH AIDS Research and Reference Reagent Program. The gene was produced from the clone by PCR employing linkers designed to match pNF1194 in AgeI-BclI sites, so as to replace the GFP. The clone Amp6-gp160-REV (pNF1221) (shown in FIG. 27) contains, in addition to the gp160 gene, the REV cDNA of F12-HIV1 subtype B from a pSV-REV clone. The clone was a gift of Prof. Jonathan Gershoni and Dr. Galina Denisova, Tel Aviv University. The REV gene was digested by XbaI and cloned into the parallel site in pNF1220, creating pNF1221. The amp6-gp160/amp6-gp160-REV constructs have the HCMV promoter and the SV40 polyadenylation signal. Plasmid DNA was prepared in E. coli DH10B or K12 GM2163 (DAM⁻/DCM⁻), using the Nucleobond AX plasmid maxi prep kit of Machery-Nagel Germany.

The amplicon-6-MUC1 clone vector—The amplicon-6-GFP (pNF1194) vector was digested by the AgeI-BclI enzymes, flanking the GFP gene. The digest was treated with Klenow enzyme to produce blunt ends. This was followed by to reduce self-ligation. A 13500 bp plasmid containing the human MUC1cDNA was cleaved in two steps generating a fragment flanked by XhoI and XmnI, containing the MUC1cDNA and polyA signal in a fragment with blunt ends. This fragment was cloned into the blunted amplicon-6GFP less GFP. Several clones were selected containing the insert in the right orientation to the HCMV promoter in the amplicon-6 vector. Sequencing reactions confirmed the resultant 9370 bp amplicon-6-MUC1 vector. As further described, the clone produced protein, which could be reacted with MUC-1 hybridoma antibody H-23.

Extraction of Total Infected Cell DNA

Total DNA was extracted from infected and non-infected cultures by using the EZ-DNA genomic isolation kit (Biological Industries co.), according to the supplier's protocol (based on the Guanidinium Isothiocynate reagent).

Extraction of Total Infected Cell RNA

Total RNA was extracted from infected and non-infected cultures by using the EZ-RNA Total RNA Isolation Sample kit (Biological Industries co.), according to the supplier's protocol (based on the Guanidinium Isothiocynate reagent for denaturation, phenol and chloroform for extraction and protein removal).

Protein Assay from Transfected Cells

Transfection-Infection (Superinfection) Assay

24 h before electroporation the J-JHAN cell were passaged, and then washed twice in PBS (without calcium and magnesium). The washed cells were resuspended in RPMI-1640 medium at 1×10⁷ cells/ml. 0.4 ml of the cells were mixed with 50 μl of purified plasmid DNA in 4 mm gap disposable cuvette (BTX P/N 640), and then electroporated by one pulse of 250 V, 24msec (Electro cell manipulator ECM 395). The electroporated cells were incubated for 10 min on ice and then transferrto 5 ml of RPMI 1640 medium supplemented with at 10% fetal calf serum and 50 μl/ml gentamicin at a final dilution of 4×10⁶ cells/ml. 24 h-48 h after electroporation, the cells were infected with concentrated aliquots of the HHV-6 U1102 strain infected cells. Viral cytopathic effects peaked usually 5 to 8 days after infection, and the electroporated/infected cells were harvested for protein extraction.

Protein Extraction

The superinfected cells were washed twice with PBS without calcium and magnesium (Biological industries), then lysed by adding 200 μl of 4° C. lysis buffer. The lysed cells were rotated at 4° C. for 1 h, and centrifuged in an eppendorf centrifuge 20′-30′ at 14000 rpm. The supernatant was collected into new eppendorf tubes and frozen at −70° C. Aliquot of the extracted proteins were measured to determine the protein concentration, by the Bradford method, using a 96 well ELISA reader at 595 nm wavelength. BSA was used for calibration.

Protein precipitation with TCA To precipitate the proteins secreted to the medium, 24 h -48 h before precipitation the cells medium was replaced to Bio-Ram-1 medium (protein free). The cells were pelleted by centrifugation 5′ at 2000 RPM. The medium was filtered through 0.45 μm filter. To each 0.5 ml fraction (in eppendorf tube) 1 μg of BSA was added as a carrier. 125 μl of 50% TCA was added and mixed, to give final concentration of 10% TCA. The mixture was incubated 10′ at −20° C., and then spun in a microcentrifuge at 4° C., top speed for 20-30min. The supernatant was carefully removed and the pellet was resuspended in 12-20 μl of 1×loading buffer with β-mercaptoethanol. If the color was yellow, (indicating acidity), 0.5-7 μl of 1M Tris pH 8.0, were added till the color turned blue.

Protein Electrophoresis

The electroporated/infected cells were harvested and lysed in 50 mM Tris HCL (pH7.5), 150 mM NaCl, 0.5% NP-40 and protease inhibitors (Complete protease inhibitor, Roche). Protein samples were first denatured by 5′ boiling and β-mercaptoethanol (Sigma), in the loading buffer, and then were loaded on 8-12% Tris-Glycine SDS-polyacrylamide gels, in the Bio-Rad running device, employing running buffer at constant current of 40 MA per gel. A molecular weight protein marker was used. The gel was transferred to nitrocellulose membrane (Schleicher & Schuell), washed 3 times in ddH₂O and stained with a gel code blue stain reagent.

Western Blotting

The proteins that ran on the Tris-Glycine SDS-polyacrylamide gel were transferred to nitrocellulose membrane by attaching the gel to the membrane and by pressing with Whatman paper and Dacron sponges from both sides. The cassette was placed inside the transfer device (Bio-Rad), in transfer buffer with an ice vial, at a constant voltage of 60V for 2h-3h.

Immunoblotting

The nitrocellulose membrane with the transferred proteins, was blocked with 5% milk in TTBS for 1 h at R.T, then, rinsed once briefly with TTBS and incubated for 2 h RT. with a primary antibody diluted between 1:200 to 1:5000, in TTBS containing 1% BSA and 0.05% Sodium Azid (NaN3). The membrane was washed 4 times, 5′ each, with TTBS, and then incubated with the secondary antibody Goat anti-mouse IgG peroxidase-conjugated affinipure diluted 1:5000 to 1:25000 in 5% milk in TTBS, (Jackson Immunoresearch Laboratories). Following incubation with the secondary antibody for 45′ -60′ at R.T, the membranes were washed 4 times 5′ each with TTBS. Enhanced chemiluminescence (ECL) mixture (SuperSignal West Pico Chemiluminescent Substrate (ECL)-Pierce), was added to interact with the horseradish peroxidase (HRP), the tag on the secondary antibody, causing light emission, detected on Scientific imaging X-OMAT Kodak film.

Detection of GFP in Lymphocytes

300 μl samples of cells were washed once with PBS and resuspended in 1/10 volume of PBS. The concentrated cells were placed on glass slides that were coated with poly-L-lysine (1 mg/ml). The cells were fixed with 4% paraformaldehyde for 15′-20′ R.T, and washed with PBS. Then Galvanol was added and covered with cover slip. The fluorescent cells were visualized using fluorescence Axioskop microscope (Carl Zeiss, NY) and camera photographs were taken using 200ASA color films (MC-100 camera).

Immunofluorescence Studies in Lymphocytes

300 μl samples of cells were washed once with PBS and resuspended in 1/10 volume of PBS. The concentrated cells were placed on glass slides that were coated with poly-L-lysine (1 mg/ml). The cells were fixed with 4% paraformaldehyde for 15′-20′ RT, washed 1′ 3 times with PBS, before the addition of 0.1% TritonX-100 and 10′ incubation at R.T. For immunofluorescense detection inside the cell, this step was optional. The TritonX-100 was washed twice for 5′, and the cells were then exposed for 30′ at R.T to 20% fetal calf serum in PBS for blocking. The cells were then incubated for 30′ at R.T with a primary antibody at 1:200 dilution, rinsed 3 times with PBS at R.T for 10′ with gentle shaking. Secondary antibody, (1:500 Goat anti-mouse IgG Rodamine-conjugated) was added for 30′ at R.T. Cells were then washed with PBS 3 times for 10′ at R.T., with gentle shaking. At the end Galvanol mounting reagent at 100 mg/ml Mowiol (Calbiochem, LaJolla, Calif.) was added and the cells were covered with cover slip. The cells were viewed with an Axiovert 135M confocal microscope (Carl Zeiss, NY) equipped with an argon-krypton laser using a 100× objective lens; excitations were at 488 and 568 nm. Contrast and intensity for each image were manipulated uniformly using Adobe Photoshop software.

PCR Amplification

Generally 200 ng-1 μg DNA as a template or DNA from bacteria colonies were taken by picking. The prepared reaction mixture contained 10 μM of each primer, 2.5U Taq polymerase with standard buffer conditions (MgCl₂, 1.5 mM final concentration) and 10 mM of the deoxnucleotide triphosphates (2.5 mM each), in a total of 50 μl per reaction. The PCR amplification reaction profile was usually one cycle of 5′ at 94° C. followed by thirty cycles of denaturation for 1 min at 94° C., annealing for 1 min at 50° C.-65° C. (depending on the primer's annealing temperature), and extension for 1-3 min at 72° C. (depending on the amplified section length). Following the 30 cycles, an additional 10′ at 72° C. needed for complete polymerization, and cooling to 10° C. till the samples were carried out from the PCR. The PCR amplification reactions were done in a DNA thermal cycler (TechGene, Techne). The amplified products were then electrophoresed in 1% agarose gels with ethidium bromide staining. The products were gel extracted and cloned into the appropriate vector as detailed in “Results”.

Sequences of DNA oligonucleotide primers used:
gD sense:
5′-CAG CTT CAC GAC CGG TAG GTC TCT TTT GTG TGG
TGC-3′
gD anti sense:
5′-GAT ACT AGC CTG ATC AGG GGT ATC TAG TAA ACA
AGG-3′
gD sec anti sense:
5′ACT AGC CTG ATC ACT AGG CGT CCT GGA TCG ACG
G 3′
gD sequence 301
5′-GAG GCC CCC CAG ATT G-3′
gD sequence 639
5′-CTG TAA GTA CGC CCT CC-3′
gD sequence 3151
5′-GTA ACA ACT CCG CCC CAT-3′
gp160 short sense
5′-GTG GCA ATG AGA GTG AAG-3′
gp160 short anti sense
5′-CTA TAG CAA AGC CCT TTC C-3′
gp160 long sense
5′-CAG CTA CCG CTG GCC GGC CAG GCC TGT GCA GCG TAC
GGT GGC AAT GAG AGT GAA GGA G-3′
gp160 long anti sense
5′-GAT ACT GAT CAG GCC ATT CAG GCC TTC GAA CGT ACG
CTA TAG CAA AGC CCT TTC CAA AC-3′
gp160 sequence 402
5′-GGA GAA TAG TAC TAA TGC C-3′
gp160 sequence 1024
5′-GAC ACC TTA GGA CAG ATA G-3′
gp160 sequence 1705
5′-CAG CTC CAG GCA AGA ATC-3′
gp160 sequence 2356
5′-GCC CTC AAG TAT TGG TGG-3′
REV HXB2 s 5970
5′-GGA TTG TGG AAC TTC TGG -3′
REV HXB2 as 6030
5′-GCT TGA TGA GTC TGA CTG-3′
Rev promoter 603
5′-GTT CGG CTG CGG CGA G-3′
CMVp sequence
5′-GTA CGC GGG GCT AGA GCG-3′
CMVp end seq
5′-GTA ACA ACT CCG CCC CAT-3′

The DNA oligonucleotide primers were synthesized using a DNA synthesizer (Sigma).

Reverse Transcriptase (RT)-PCR Reaction

Samples of total RNA from infected and uninfected cells were used for RT reaction (with Expand Reverse Transcriptase kit, Roche). Reaction included 5 μg of total RNA, 100 pmols oOligo (dT)₁₅ and sterile RNase-free H₂O up to 31 μ1. The mixture was incubated 10′ at 65° C. and placed on ice for 2′. The other reagents were added to the same tube as follows:

4 μl of 5*Expend reverse transcriptase buffer

2 μl of 100 mM DTT

8 μl of dNTPs 10 mM each

20 units of RNase inhibitor

50 units of Expend Reverse transcriptase

The reaction was incubated for 1 h 42° C. and then for 5′ at 95° C.

The cDNA (cooled to 4° C. and stored at −20° C.), was used as a template (1 μl-5 μl), for regular PCR amplification reaction.

Confocal Microscope Analyses

To determine the location of expressed gD and gDsec, gp160+REV proteins, and MUC1 tumor antigen in the various experiments in the cells, cell samples were concentrated, rinsed with PBS and placed on glass slides coated with poly-L-lysine (1 mg/ml). After fixation with 4% paraformaldehyde, cultures were perforated with 0.1% TritonX-100 and rinsed with PBS. The slides were blocked with 20% fetal calf serum in PBS to reduce background, and then incubated for 30′ with the appropriate antibodies. For gD and gdsec expression, slides were incubated with gD H170 antibody. For gp160 expression, slides were incubated for 30′ with soluble CD4, followed by incubation with CG10 (mouse mAb IgG antibody) known to interact with the gp120-CD4 complex (gift of Prof. Gershoni, Tel-Aviv University). Slides were then incubated with secondary Cy3- or FITC-conjugated Goat anti-mouse IgG (Jackson ImmunoResearch Laboratories). After PBS rinsing the slides were covered with coverslip and Galvanol mounting reagent [100 mg/ml Mowiol (Calbiochem, LaJolla, Calif.) prior to viewing in an Axiovert 135M confocal microscope (Carl Zeiss, NY) equipped with an argon-krypton laser using a 100× objective lens. Excitation was at 488 and 568 nm. Contrast and intensity for each image were manipulated uniformly using Adobe Photoshop software.

### EXAMPLES

### Example 1

### Preparation of the Amplicon-6 and Tamplicon-7 Vector System

The plasmid pEGFP-C3 of Clontec contained a multi-cloning site linker at the C-terminus of the GFP gene, designed for fusion proteins. The linker was removed and the coding region was cloned in Bluescript—SK.EGFP (pNF1193). Then, a fragment containing both the cleavage and packaging signals and the origin of HHV-6 replication (r-pac/orilyt fragment) was cloned in pNF1193 and the final construct was designated Amp6-GFP (pNF1194). Note that Amp6-GFP has the CMV promoter between the PstI and AgeI, driving GFP, and the poly A bounded by the BclI. From this vector, the GFP gene may be replaced by any DNA sequence of interest, including the gD, gDsec, gp160 and MUC1 (see ix plsmid cloning. In each case the DNA sequence is obtained by PCR of a clone containing the desired DNA sequence, using primers, which carry in them the matching AgeI and BclI sites (see Materials and Methods).

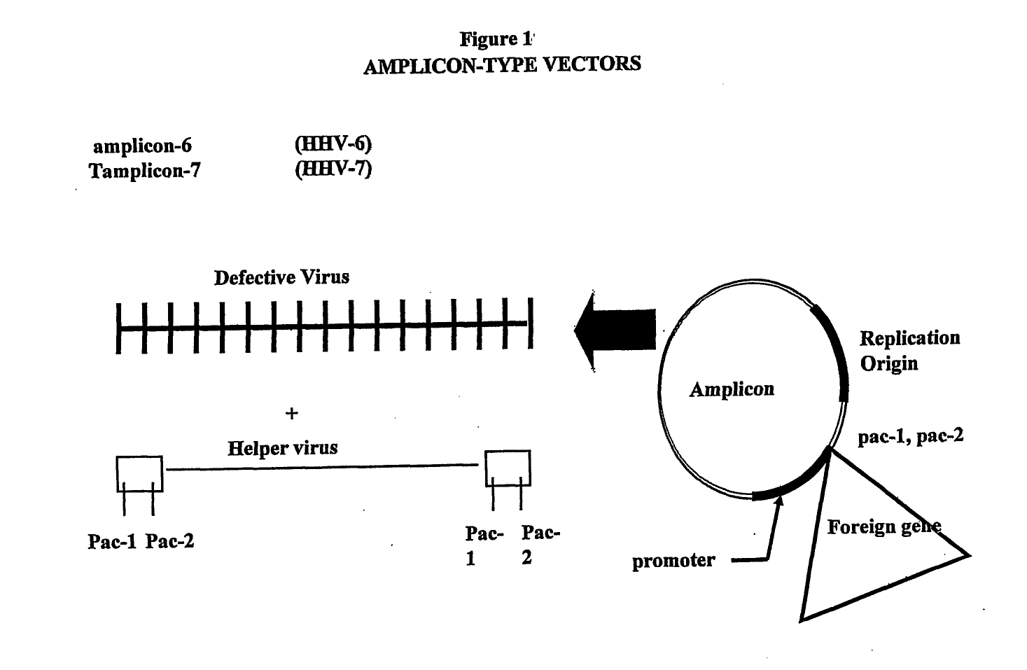

FIG. 1 shows the amplicon-type vectors, amplicon-6 and Tamplicon-7. The amplicon-type vectors contain a DNA replication origin, the pac-1 and pac-2 packaging signals and a site to insert at least one DNA sequence (e.g. as in Romi et al., 1999). A rolling circle replication of the amplicon plasmid using enzymes and functions contributed by the helper virus yields defective virus genomes with multiple reiterations of the input amplicon plasmids. The concatameric genomes are packaged in virions contributed by the helper virus.

### Example 2

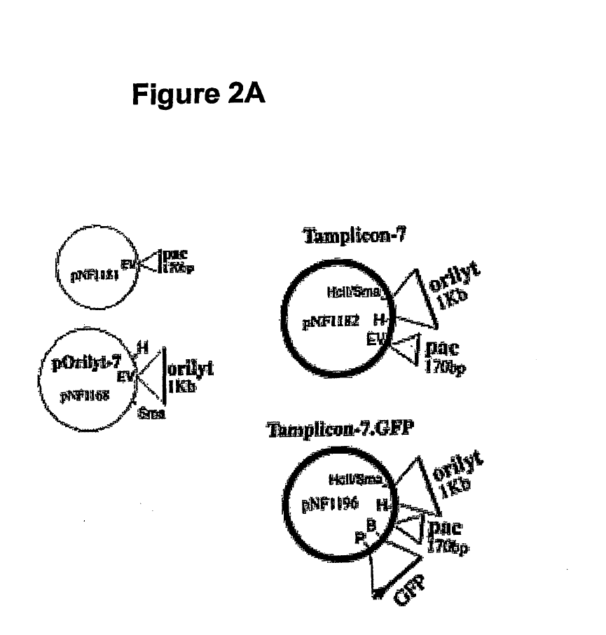

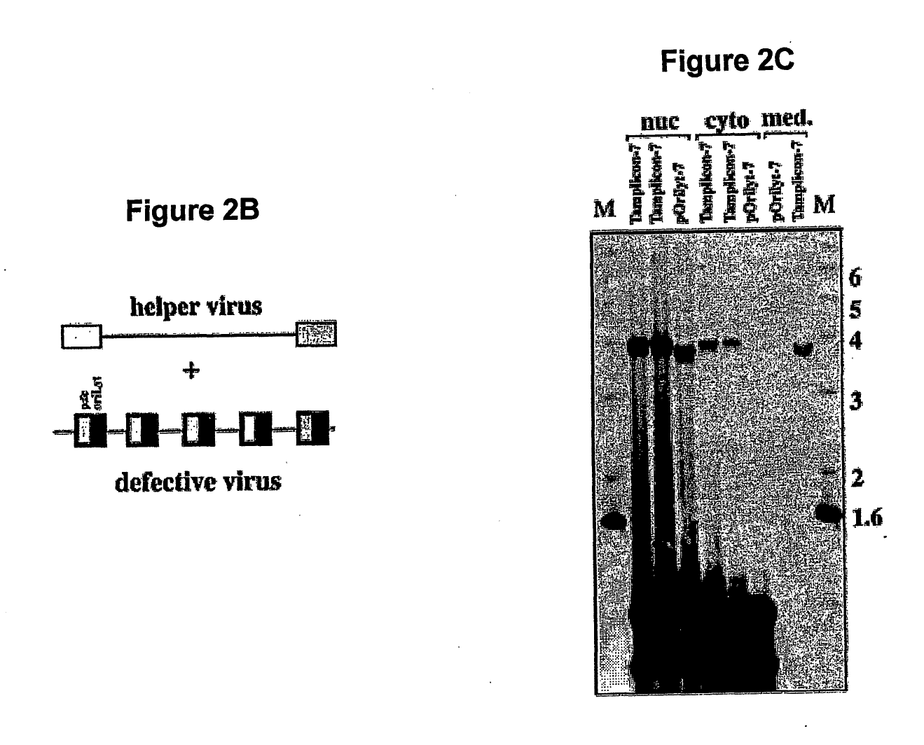

### Tamplicon-7 Vector with the Green Fluorescent Protein (GFP) Marker (FIGS. 2A-C).

The 1.6-kb GFP gene was excised from the pEGFP plasmid (Clontech) and ligated to pNF1182 between the BamHI and PstI sites. The resulting plasmid was designated Tamplicon-7.GFP (pNF1196). The GFP gene is driven by the Human Cytomegalovirus (HCMV) promoter.

Two independent infected cultures were electroporated with Tamplicon-7 and a third culture was electroporated with pOrilyt-7. Nuclear (nuc) and cytoplasmic (cyto) DNA preparations and DNA from purified virions prepared from the medium (med.) were extracted from all three cultures, digested with XhoI and DpnI, and with a GFP probe. In the presence of helper virus, the amplicon replicates by the rolling circle mechanism, yielding long, defective genomes with concatameric amplicons (FIG. 2B). pOrilyt7 are unable to exit from the nucleus, hence, the pac signals (packaging signals) are needed to exit from the nucleus into the cytoplasm, and out into the medium. In contrast to pOrilyt7, Tamplicon-7-GFP has no problem exiting from the nucleus to the cytoplasm, and to the medium (FIG. 2C).

### Example 3

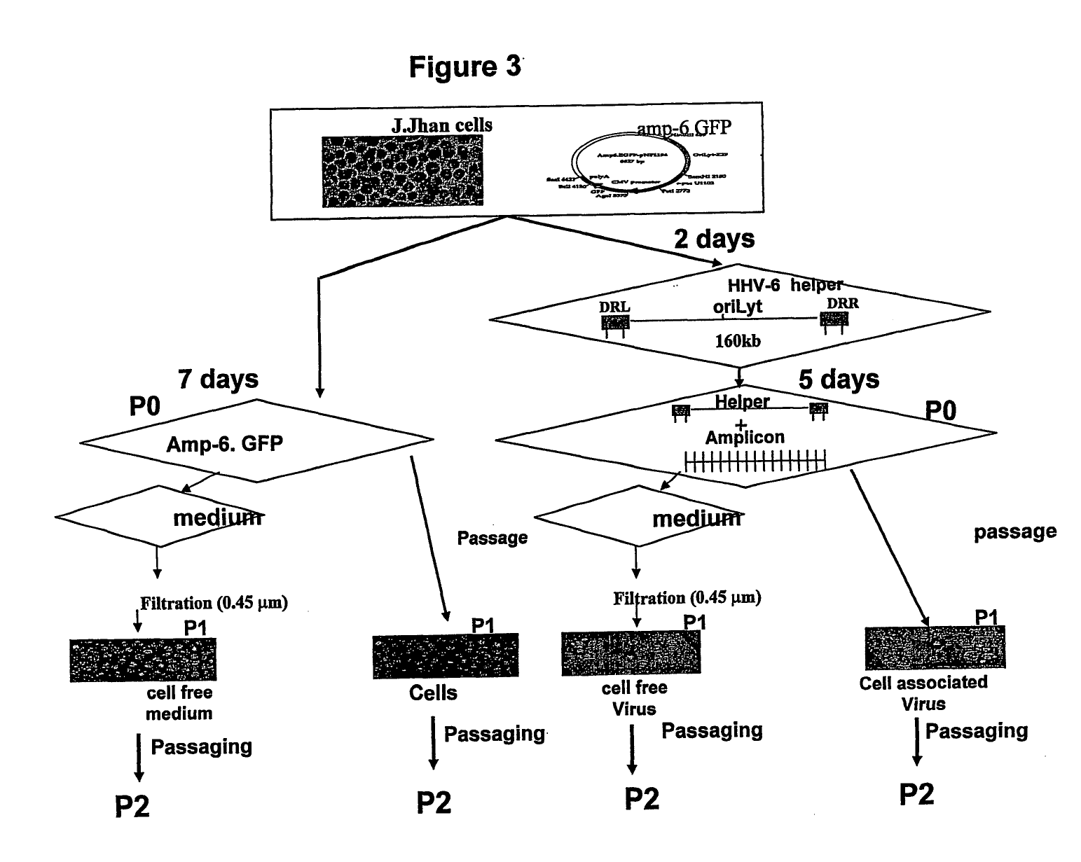

### Schematic Diagram of Cell-Associated or Cell-Free Amplicon Vectors (FIG. 3).

J-JHAN human T cells were transfected by electroporation with the amplicon-6 vector containing the GFP marker (amp-6-GFP,. Two days later (right arrow) a portion of the cells were superinfected with the helper virus HIV-6A strain U1102. The transfected/superinfected cells (Passage 0) were examined for GFP expression and were passaged by addition of uninfected cells, producing passage 1 vectors. Vectors secreted into the medium at P0 were collected by filtration through 0.45 μm membranes, to produce cell-free virions, which were further passaged in uninfected cells, producing cell-free passage 1 vectors. The analysis and passaging were repeated by adding new cells, producing passage 2 viruses. The electroporated/superinfected cells could be passaged repeatedly.

As a control served the remaining electroporated cells (left arrow) which were not superinfected with a helper virus. These cultures were handled similarly to the superinfected cultures.

As can be seen, only in cultures superinfected with the helper virus was there successful propagation of the amplicon virions. The virions were present both as cell-associated viruses and as cell-free viruses.

### Example 4

### Expression of EGFP in T cells (FIG. 4).

FIGS. 4A-4D shows fluorescent microscope micrographs of J-JHAN human T cells that were transfected with the plasmid Amp-6-GFP, prepared as above. Incubation continued with or without superinfection with the helper virus HHV-6A U1102, followed by passaging into new, uninfected cells.

FIG. 4A shows Passage 0 J-JHAN cells that were transfected with Amp-6-EGFP. Some of the cells express GFP as can be seen by the green fluorescence. Passage 0 cells were then superinfected with the helper virus (FIG. 4B), which resulted in the production of large genomes containing multiple repeats of the GFP amplicon. Indeed, many more cells expressed GFP, and the expression level was higher, as can be seen by the increased fluorescence. Similar results were obtained by quantitative analysis using FACS.

The vectors can be continuously passaged as “cell associated” (FIG. 4D) or “cell free vectors” (FIG. 4E), by filtering the medium of the lymphocytes through a 0.45 μm filters, then infecting new cells, repeatedly. Such Passage 1 cells also express GFP in high levels. In contrast the plasmid DNA with the GFP gene was expressed only in the transfected culture (FIG. 4A), but not be propagated repeatedly (FIG. 4C).

FIG. 4F shows a schematic representation of the Amp-6-GFP vector. GFP expression is driven by the Human Cytomegalovirus (HCMV) promoter.

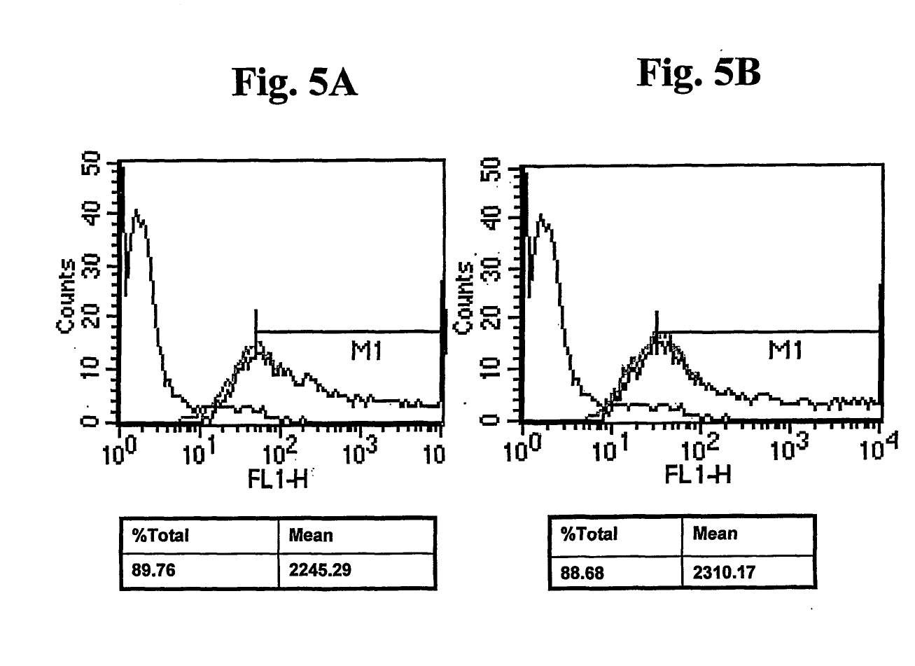

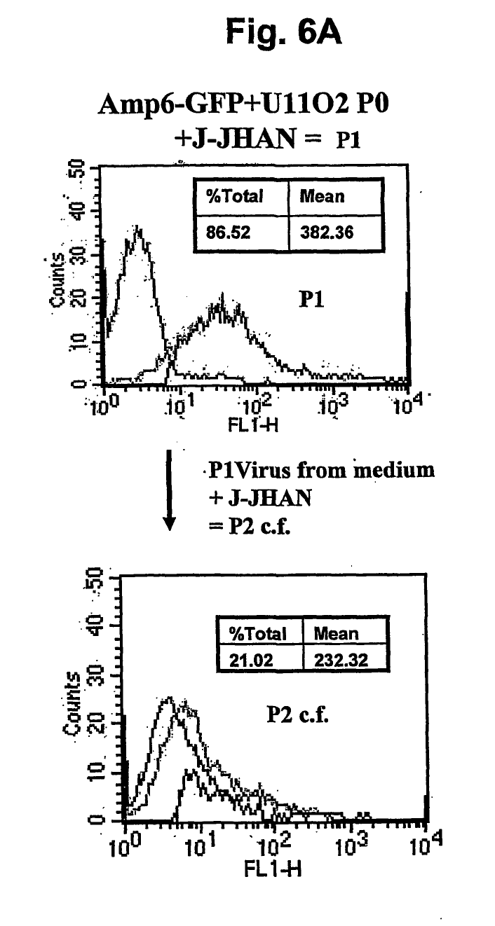

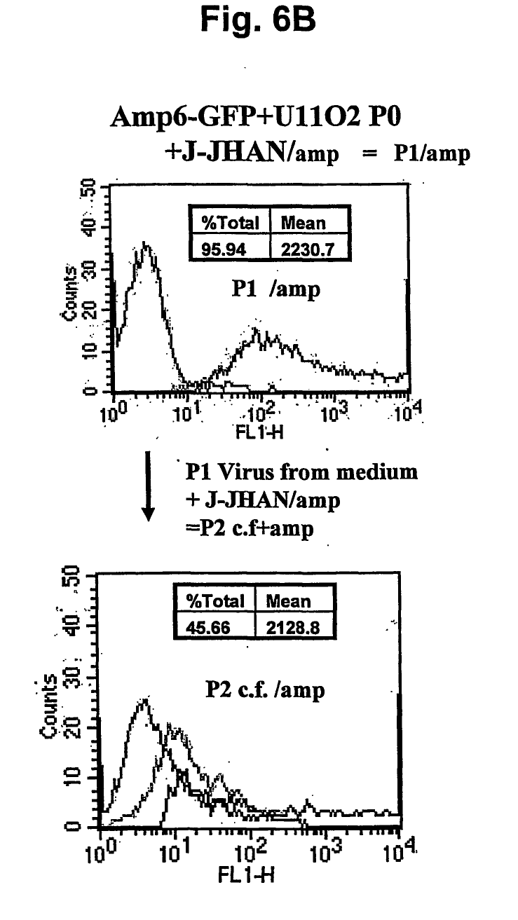

In addition to the above, expression of GFP was also quantitated by flow cytometry. FIG. 5 depicts the two identical experiments 7-day post transfection fluorescence of J-JHAN cells transfected with amplicon-6-GFP and the helper HHV-6A (U1102). FIG. 6 then shows the fluorescence at passage 1 (P1) after addition of J-JHAN cells (FIG. 6A) or J-JHAN cells (J-JHAN) or (FIG. 6B) J-JHAN cells comprising amp-6-GFP (J-JHAN/amp). Passage 2 was done, in each case, by adding cell free (c.f.) media from and the same cells. As can be seen, both in P1 and P2 the addition of J-JHAN/amp cells significantly increased the expression of GFP.

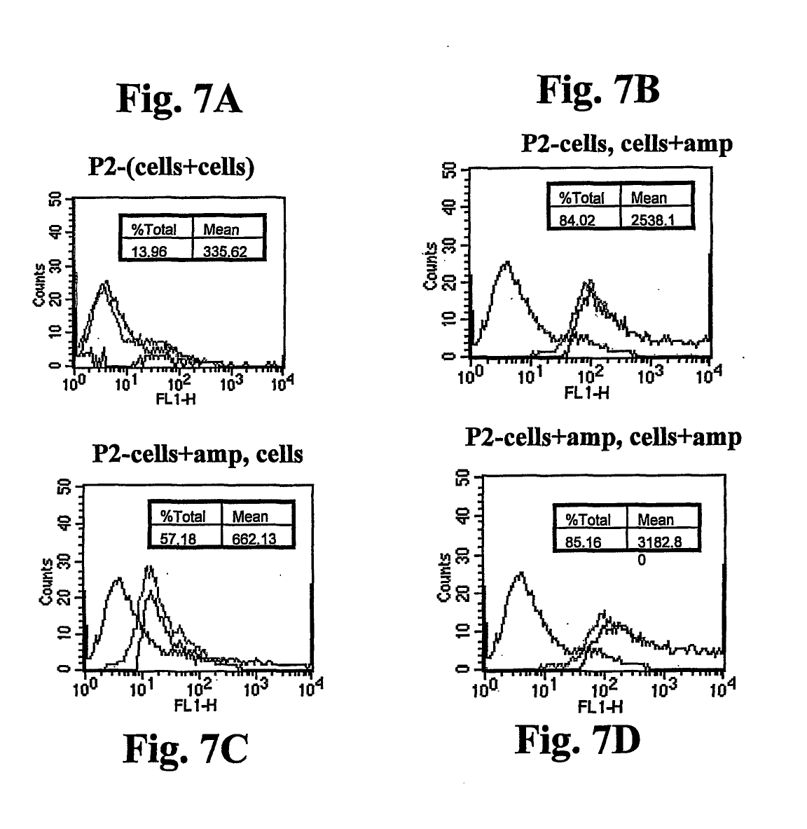

FIGS. 7A-D depict the effect of passaging on the infection capacity, as it is evident by fluorescence at P2. Four passaging combinations were used: (FIG. 7A) two passagings using J-JHAN cells (“cells”), (FIG. 7B) and (FIG. 7C) one passaging using “cells” and the other using J-JHAN cells comprising amp-6-GFP (“cells+amp”) and (FIG. 7D) two passagings using cells+amp. As can be seen one use of cells+amp (preferably for the second passaging) increased fluorescence significantly, and the best results were obtained using only cells+amp for both passaging.

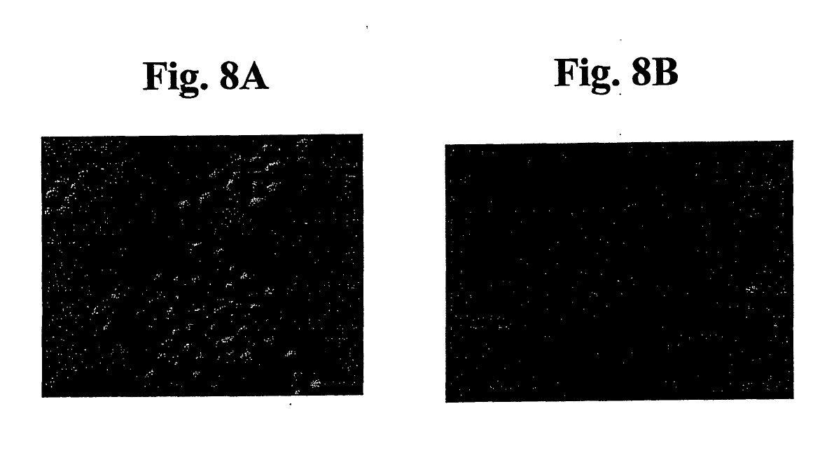

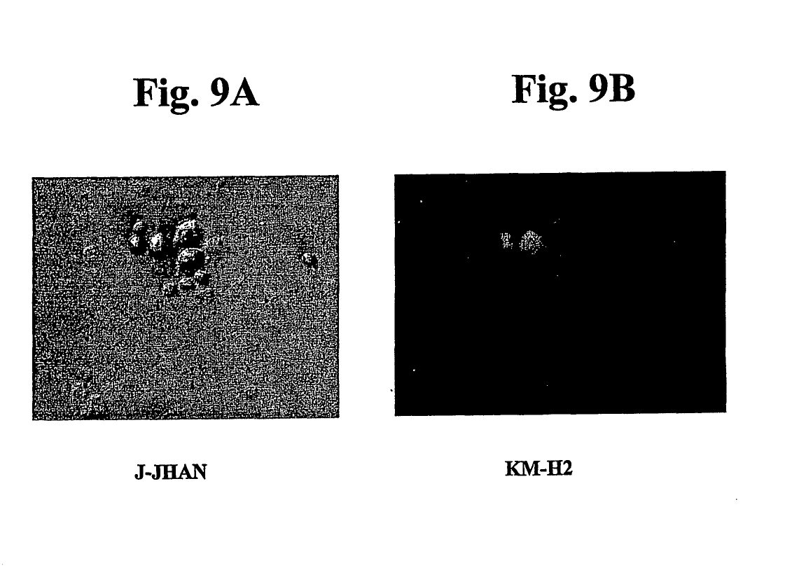

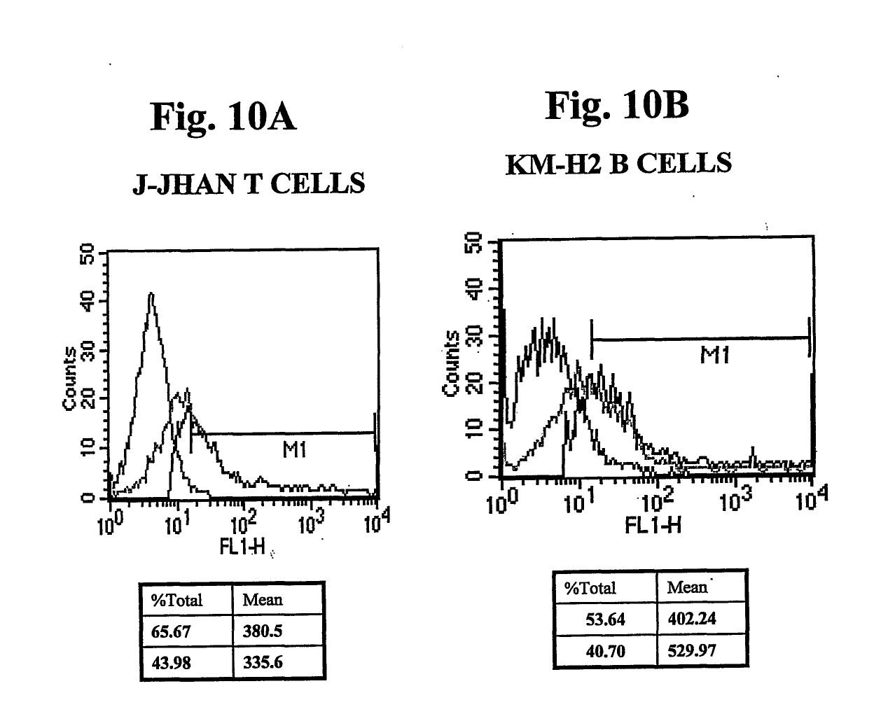

Furthermore, immunofluorescence was also used to show GFP expression in KMH-2 B cells (FIGS. 8A and 8B). As can be seen, mock transfection of the cells did not produce significant background fluorescence. However, as shown in FIGS. 9B and 9B immunofluorescence is detected (respectively) for T cells as well as B cells comprising amplicon-6.GFP. This is shown also in FIG. 10, showing corresponding flow cytometry results.

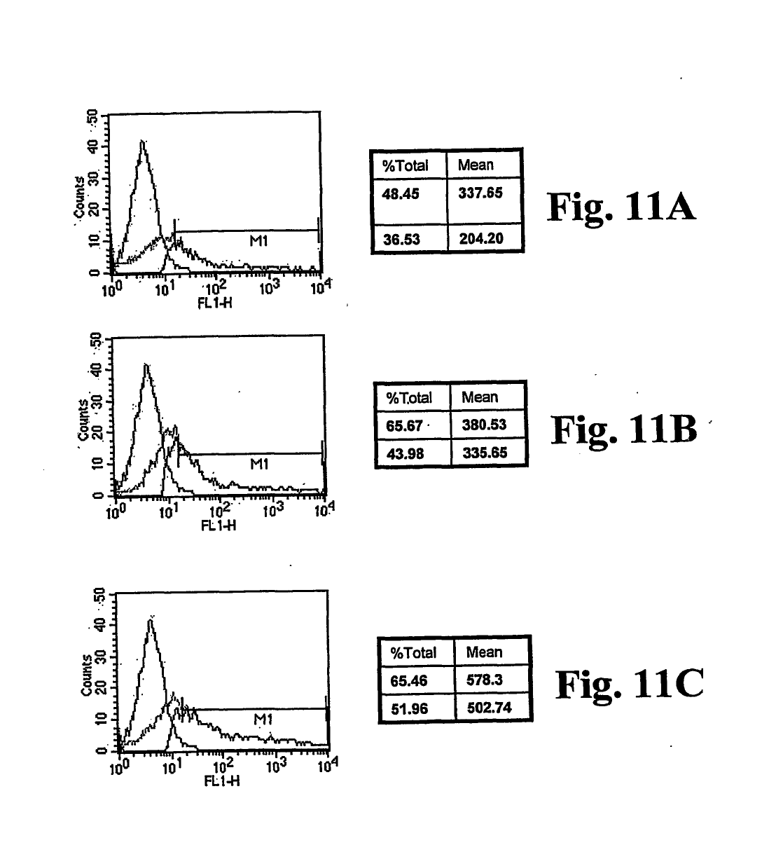

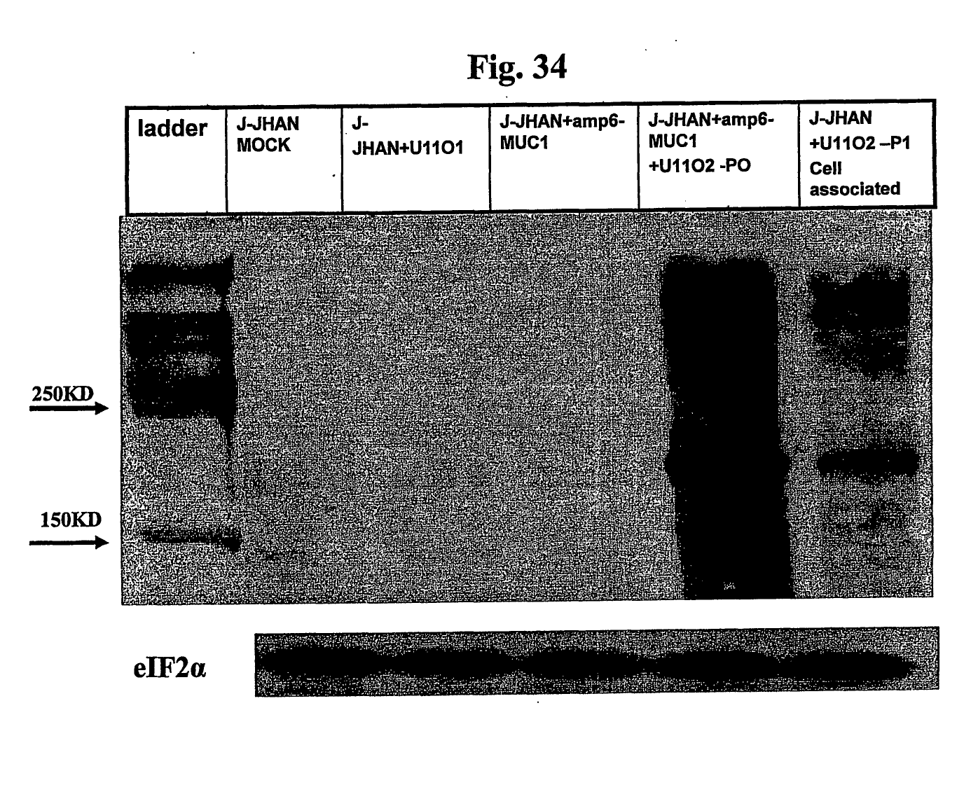

Furthermore, it was shown that the immunofluorescence is dose dependant, as shown in FIGS. 11A-C. Although the observed results do not appear to be linear, increasing the dose from 10 μl (FIG. 11A) to 20 μl (FIG. 11B) to 40 μl (FIG. 11C) also increased the fluorescence.

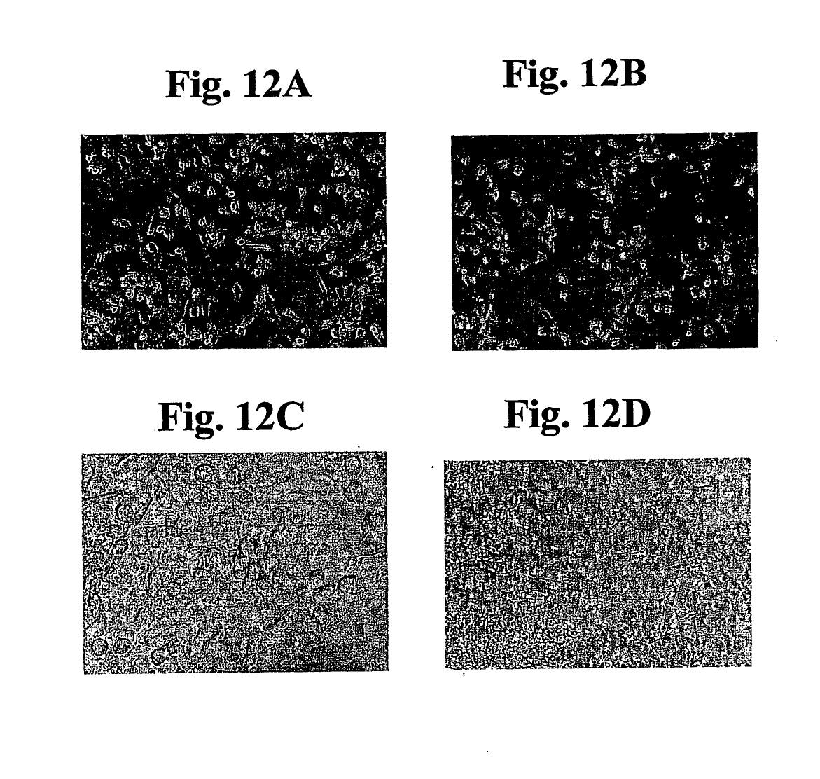

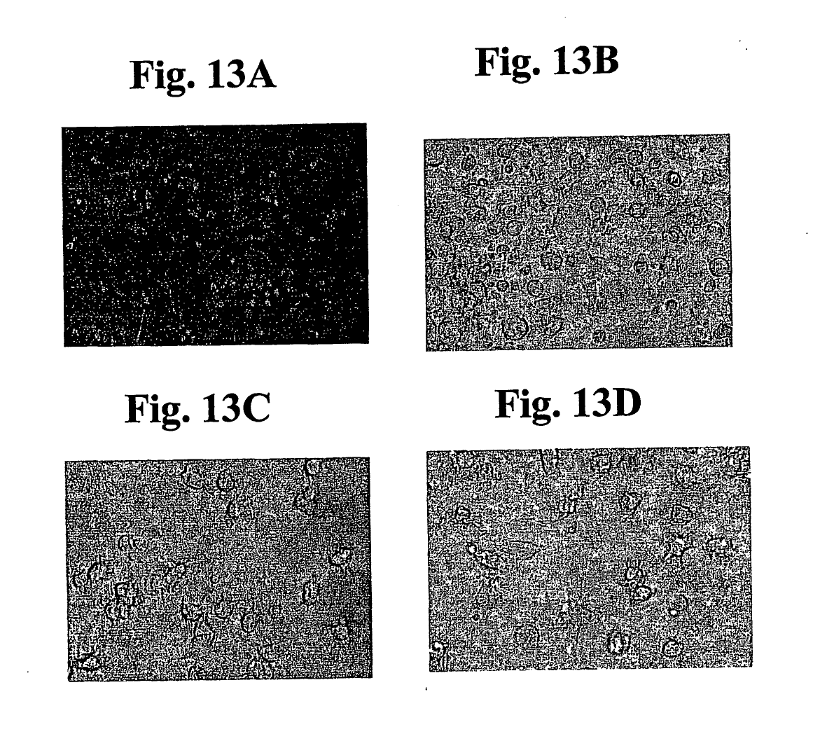

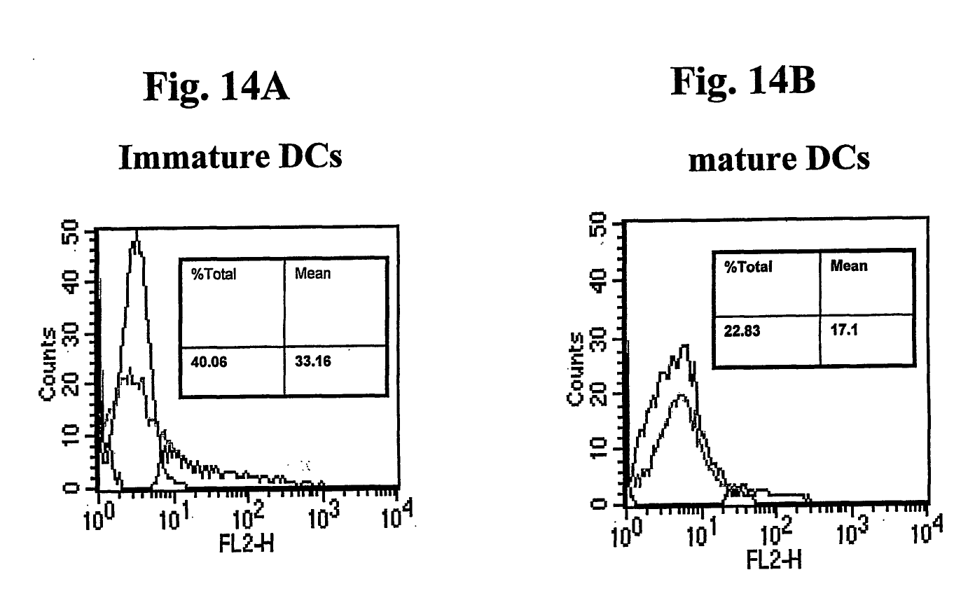

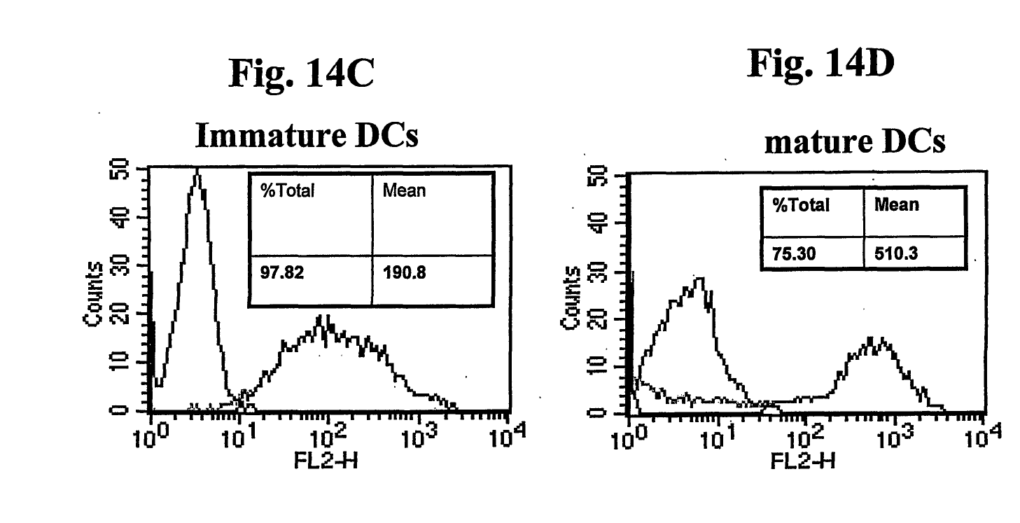

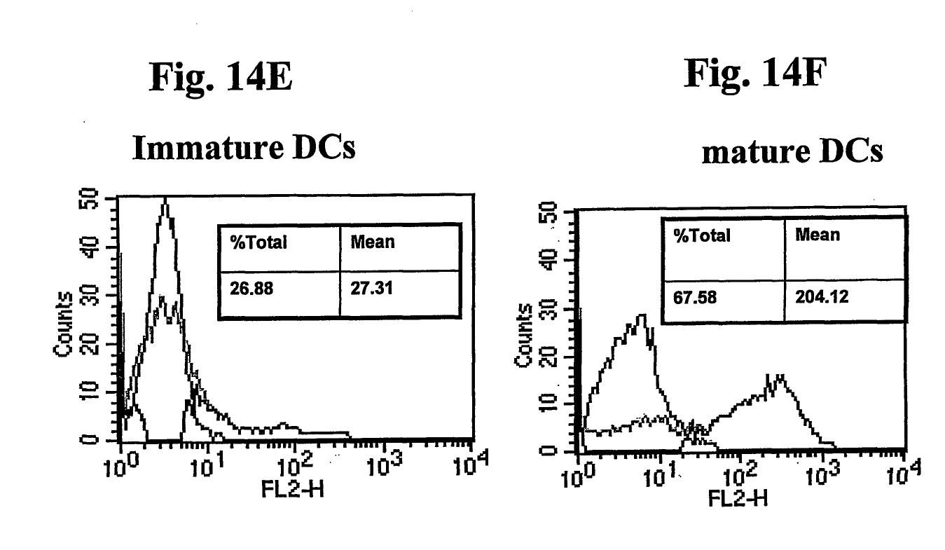

Finally, dendritic cells (DC) were also infected with amp6-GFP. Immature DC (shown in 4 samples; FIGS. 12A-12D) and mature DC (shown in 4 samples; FIGS. 13A-13D) were tested for expression of CD1A, CD86 and CD83. As seen in FIGS. 14A-14F, the expression patterns matched the maturation of DC as known in the literature.

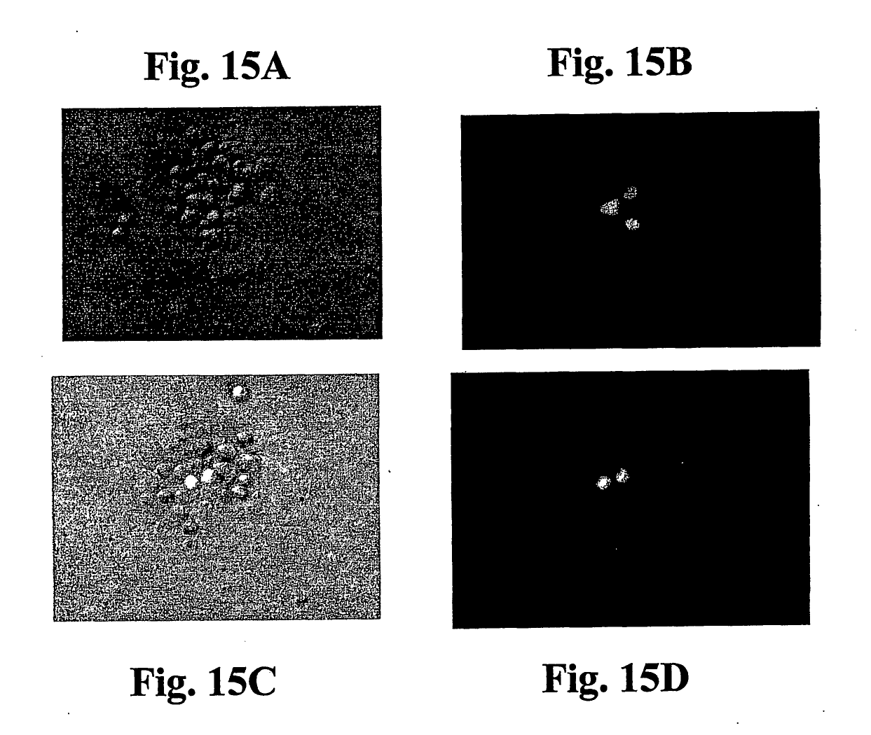

Finally, dendritic cells were infected with Amp6-GFP vector prepared as cell free virus from the medium of infected cells, and, this caused expression of GFP as seen in the fluorescent microscope photographs as seen in (FIGS. 15A-15D).

### Example 5

### Amplicon-6 Vectors Suitable for Vaccination Against HSV Glycoprotein D.

HSV-1 and HSV-2 cause painful facial and genital infections in children and adults with life long latency and repeated recurrences. Complications of the diseases are grave and include blindness and risk of fatal encephalitis in HSV infected children and adults. Furthermore, severe brain infections associated with retardation in newborn infants are due to infection by a mother with active genital herpes during pregnancy and birth.

The HSV-1 gD gene product is a major glycoprotein present in structural virions and on infected cell surface. The gene product plays an important role in viral entry and fusion to the cell membrane.

The entry of HSV into cells is an elaborate process which involves the interactions of several HSV envelope glycoproteins with an array of different receptors. HSV entry occurs by fusion of the virion envelope with the plasma membrane, and results in release of tegumented nucleocapsid into the cytoplasm. All the human entry receptors interact physically with the virion envelope component gD. The current model for HSV entry envisions fusion of the virion envelope with plasma membranes following with cell membrane interactions of four glycoproteins, gD, gB and the heterodimer gH-gL components. Cells that express gD constitutively from a transgene become resistant to infection. Due to the important role of gD in viral entry into target cells, and because of its strong immunogenic properties gD has served as a potential vaccination target.

The intact gD gene and a 201 bp deletion mutant lacking the transmembrane region (termed here as gD secreted, gDsec) were introduced into amplicon-6 (Amp6-gD and Amp6-gDsec respectively). Both genes are expressed under the control of the HCMV promoter (FIGS. 16A, 16B, respectively).

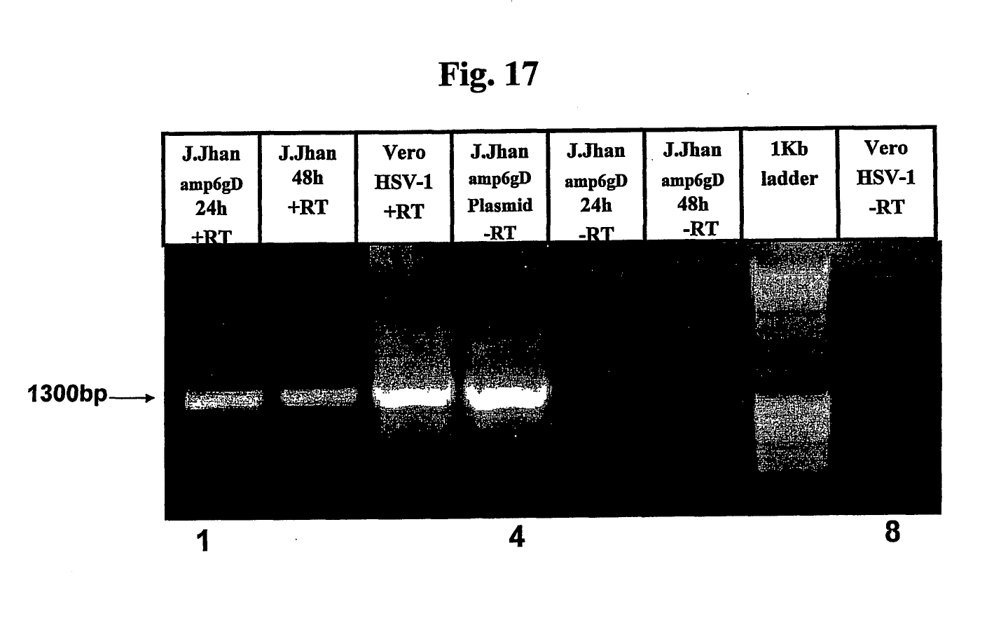

The expression of mRNA encoding gD in cells which were transfected with the Amp6-gD vector was verified. Transfected cells were used for RT-PCR analyses, employing RNA prepared 24 and 48 hours post-transfection. The RT reaction produced a cDNA product which could be PCRed yielding the 1300 bp DNA product seen in FIG. 17 (lanes 1 and 2). Control of HSV infected Vero cells also shows an identically sized RT PCR product (lane 3). Identical DNA was obtained upon PCR of a plasmid containing Amp6-gD (lane 4). No RT PCR product was produced in experiments identical to lanes 1 and 2 (24 and 48 hrs post-transfection respectively), when the RT enzyme was omitted from the reaction (lanes 5,6). Likewise the HSV infected Vero cells did not yield a PCR product when the RT enzyme was omitted (lane 8).

### Example 6

### Expression of gD and gDsec in Cells Using Amplicon-6

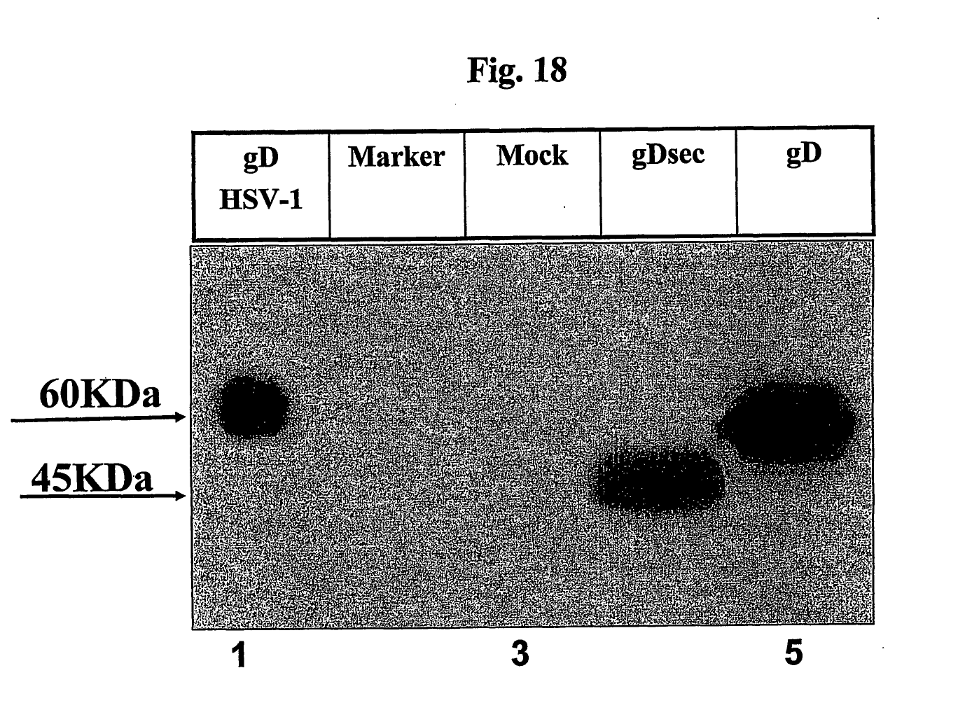

Amp6-gD and Amp6-gDsec were used to electroporate J-JHAN T cells. As controls Vero cells infected with HSV-1 were used, as well as J-JHAN cells that underwent mock electroporation. Seven days post-transfection, the cells were harvested, and analyzed by Western blotting, using anti-gD monoclonal antibodies (FIG. 18). As can be seen, the molecular weight of gDsec is smaller than that of gD, due to the deletion of the transmembrane domain.

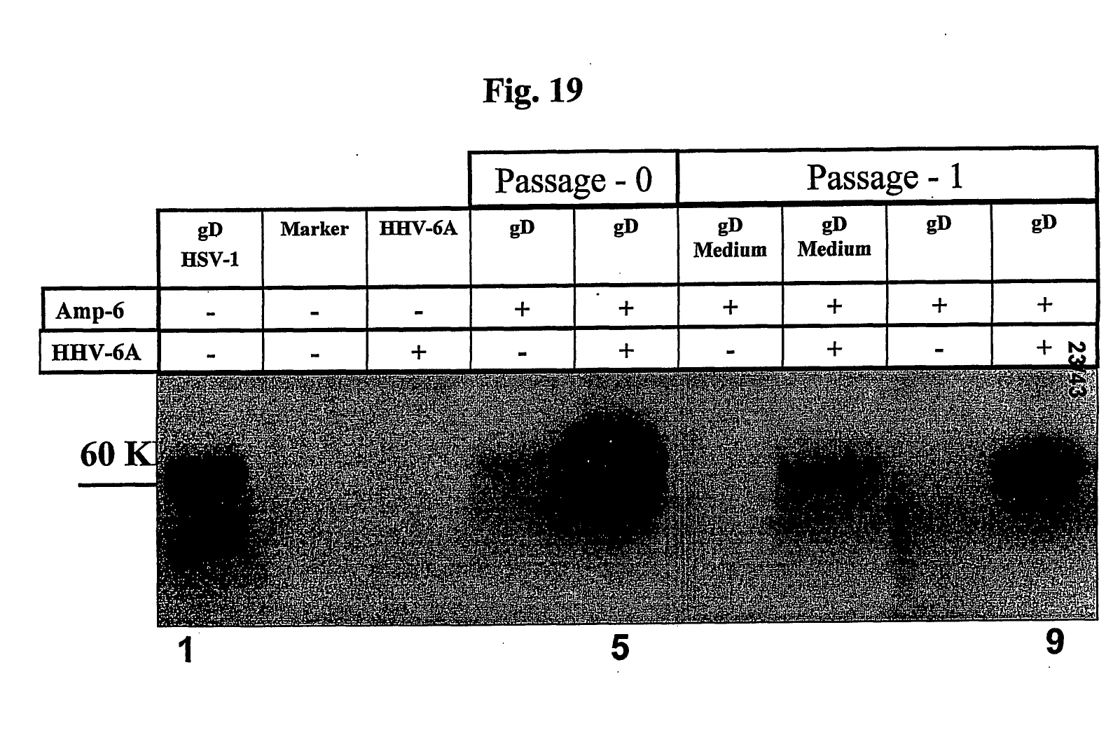

In a second experiment, J-JHAN cells were transfected with Amp6-gD and Amp6-gDsec as above, and two days post-transfection (pt.) a portion of the culture was superinfected with HHV-6A (U1102) helper virus and the rest served as control (Passage 0). Cells or the cell-culture medium were then passaged by addition of uninfected cells. Seven days later, cells were harvested at the various stages and analyzed by Western blot as above (FIG. 19). As can be seen by comparing lanes 4 and 5, expression of gD was significantly enhanced in the HHV-6 superinfected cultures. The addition of the helper virus (i.e. HHV-6) was also crucial for finding amp6-gD in the filtered medium which could be used to infect new cells (lanes 6, 7) and for expression of gD in passage 1 cells, as can be seen in lanes 8, 9: only superinfected cells expressed gD in passage 1 vector. Using the same techniques, Amp6-gD could be further passaged.

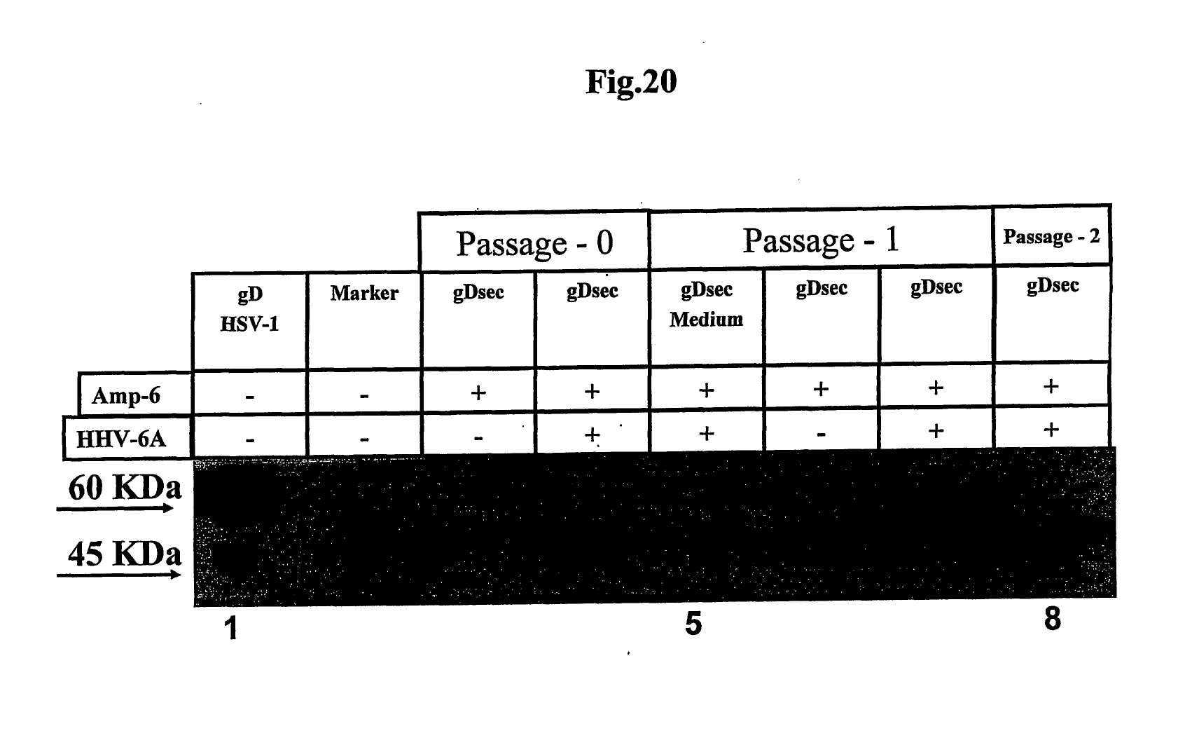

The expression of gDsec was assayed in a similar manner (FIG. 20). Cells were transfected with Amp6-gDsec and after two days were either superinfected with helper virus, or served as control. Again, the addition of the helper virus enhanced expression of gdsec (compare lanes 3 and 4 in FIG. 20), and the helper virus was necessary in order to achieve expression in passage 1 (lanes 5-7, FIG. 20). Superinfected passage 2 cells also express gDsec (lane 8, FIG. 20).

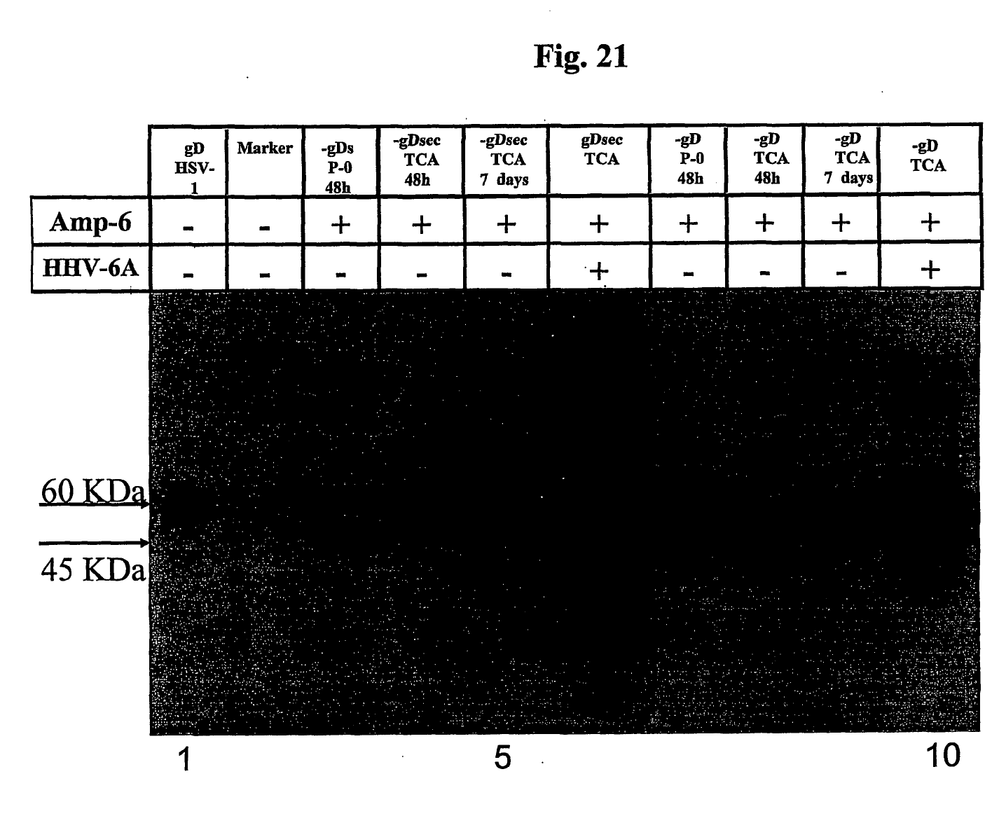

Since gdsec lacks a transmembrane domain, it is possible that it is secreted from the cells. Instead, when proteins were precipitated from the medium by the addition of trichloroacetic acid (TCA), a small amount gdsec could be detected in the medium (FIG. 21, lanes 4-5). The electrophoretic mobility was similar but not identical to the non-TCA precipitated cultures—the TCA precipitated proteins appeared higher in the gel. As can be seen, the addition of HHV-6 significantly increased the levels of gDsec in the medium. (FIG. 21, lane 6).

### Example 7

### Confocal Analysis of the Expression Pattern of gD and gDsec.

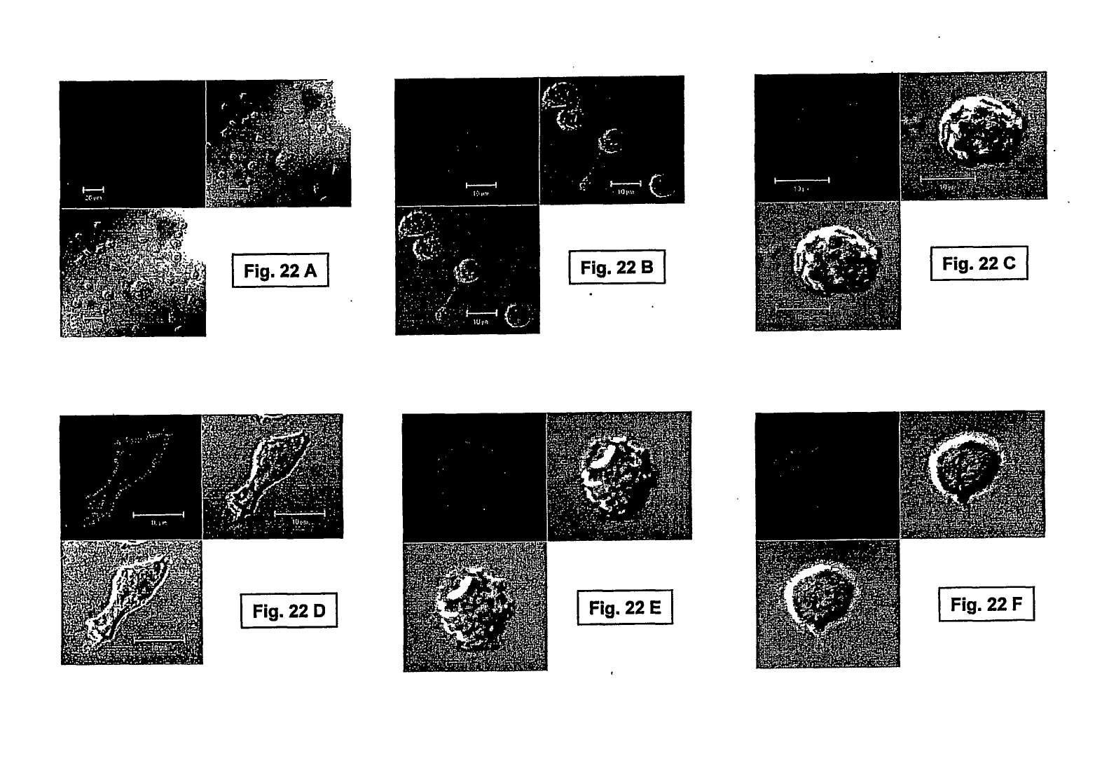

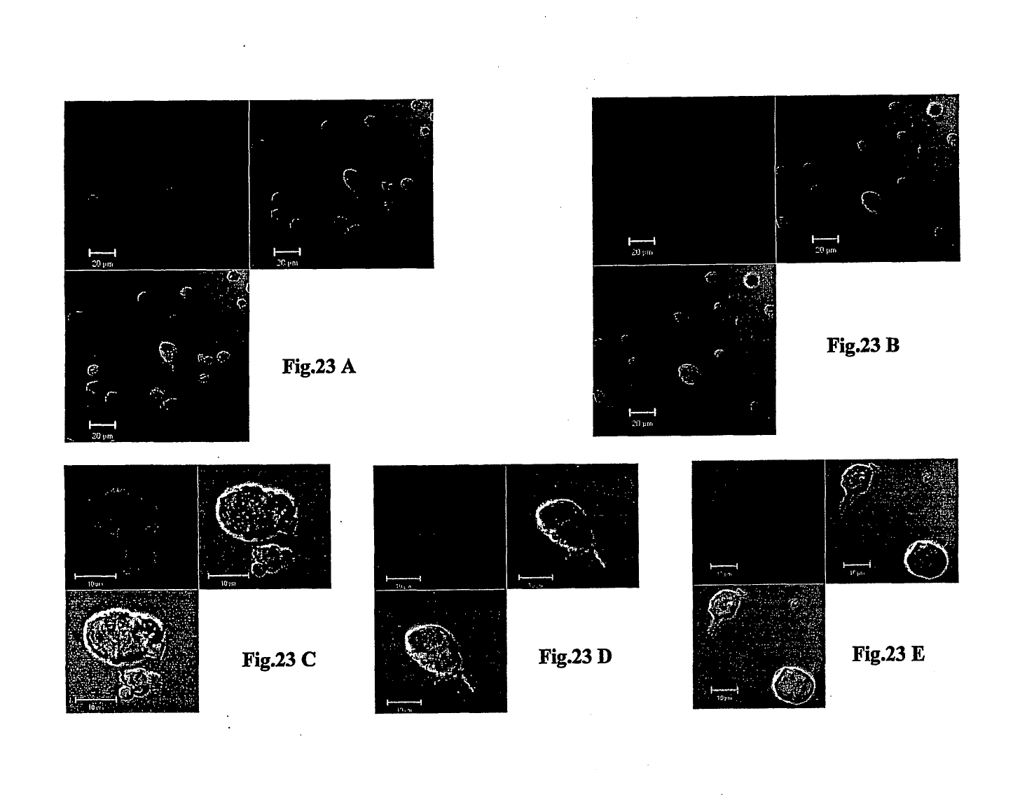

When viewed in the confocal microscope the cells infected with the intact gD amplicon produced gD protein localized preferentially at the cell surface (FIGS. 22A-22F), whereas the gDsec amplicon protein appeared to be dispersed in internal locations of the cells (FIGS. 23A-E). Altogether the experimental data regarding gD and gdsec expression demonstrate the ability to have the protein expressed efficiently in the cell surface, within the cells and as a protein secreted outside the cells.

### Example 8

### Flow Cytometry of Amplicon-6-gD Transfected J-JHAN Cells with and without Superinfecting Helper Virus

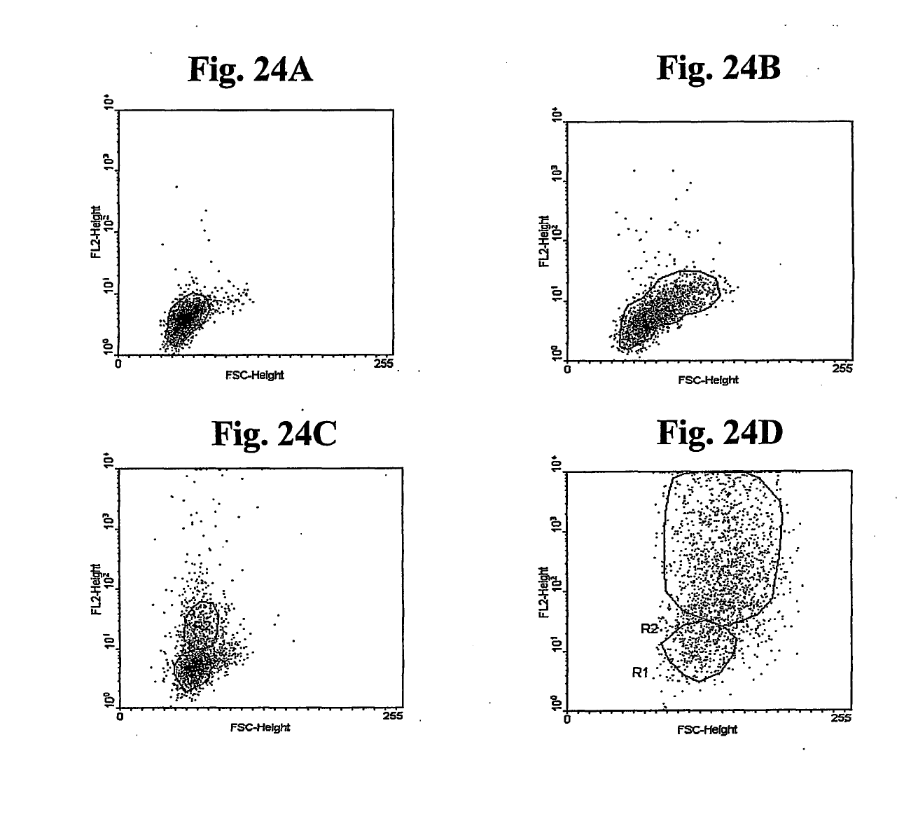

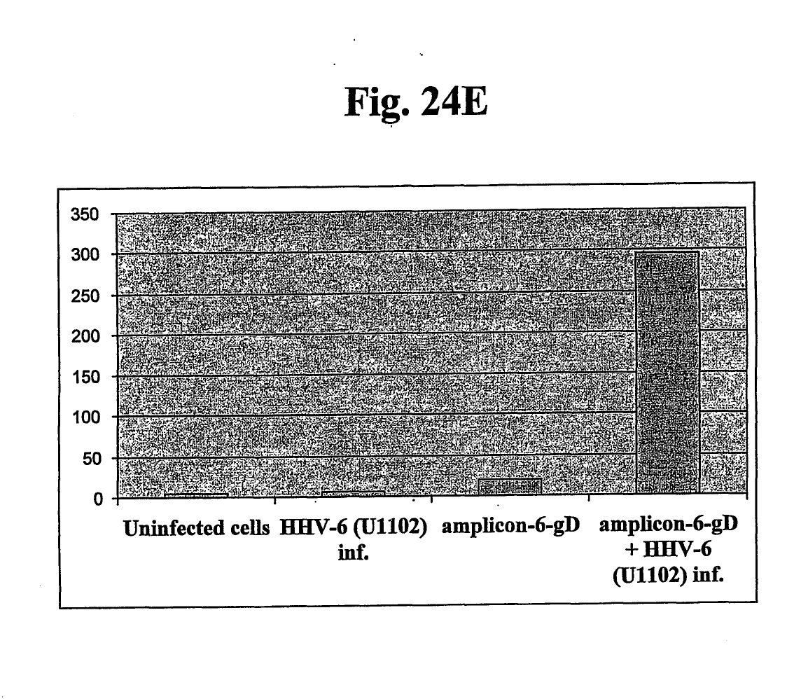

J-JHAN cells were transfected with amplicon-6-gD, either with or without superinfecting helper virus. As seen in FIGS. 24A-24D, cultures that were not infected (FIG. 24A), or infected with helper virus only (FIG. 24B) or electroporated with amplicon-6-gD only (FIG. 24C), showed very little fluoresnce, when compared with cells that received both the amplicn-6-gD and the helper HHV-6A (U1102) (FIG. 24D). The mean fluorescence intensity (MFI) of the above different cultures is shown in form of a chart in FIG. 24E.

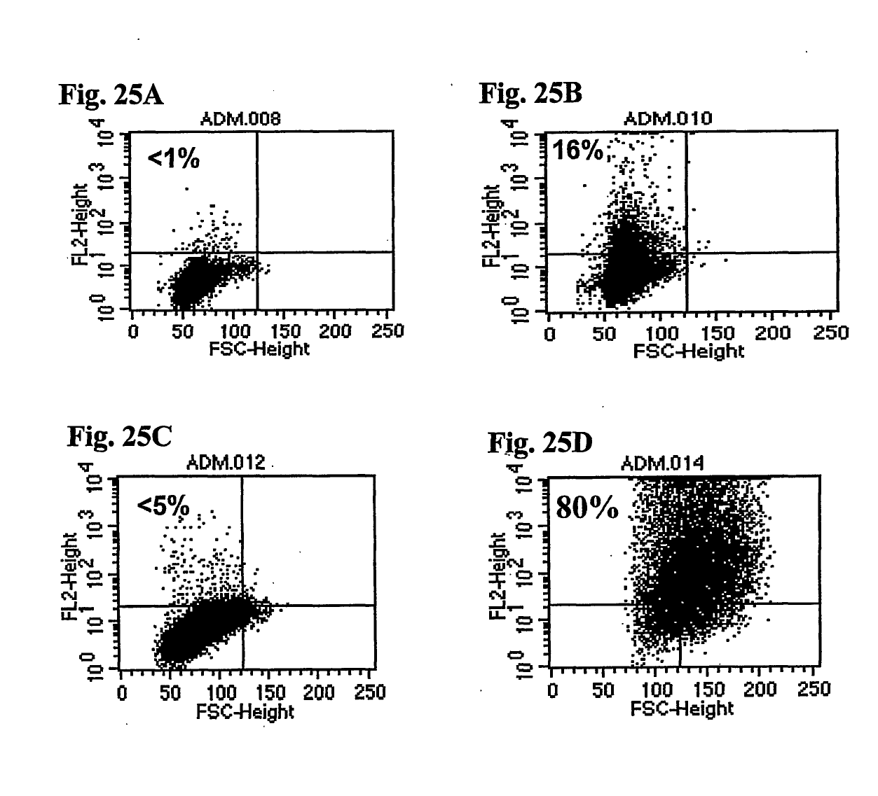

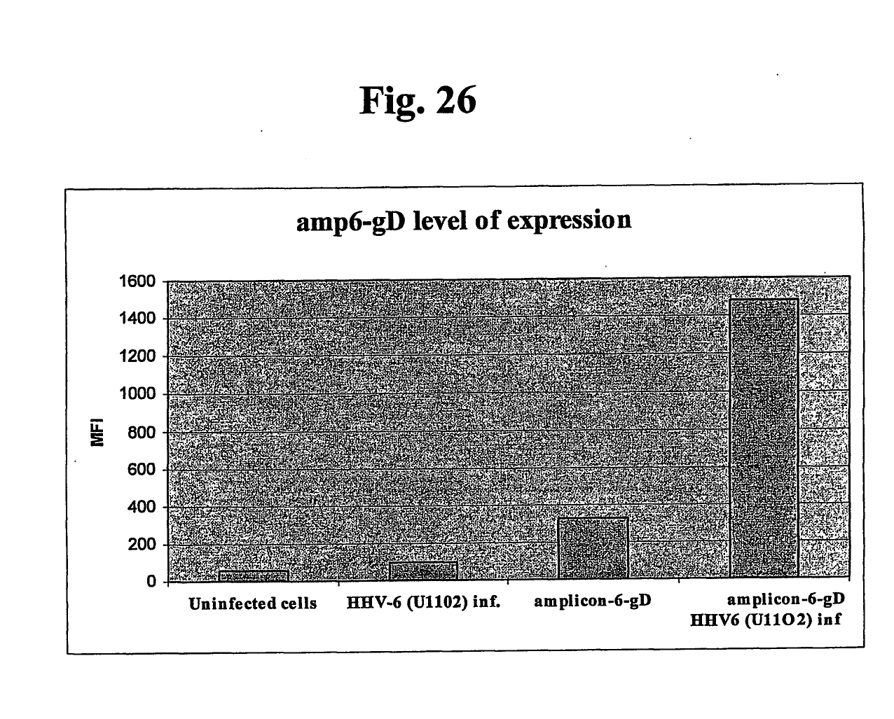

The results of a similar experiment are shown in FIGS. 25A-25D and FIG. 26. as can be seen, the highest fluorescence was observed when the cells received both the amplicon and the helper. Receipt of either one, or without infection, caused much lower fluorescence.

### Example 9

### Amplicon-6 Vectors Carrying the HIV-1 gp160 and REV.

Another example of proteins which were chosen for amplicon-6 mediated expression in T cells towards vaccination, corresponds to the envelope (env) gp160 gene of HIV and the REV gene. The product of gp160 is a 160 KDa polyprotein precursor of the proteins glycoprotein 120 and gp41 present on the virus envelope and infected cell membranes. Cleavage of gp160 is required for env-induced fusion activity and virus infectivity. The protein, which is anchored to the cell membrane, contains determinants that interact with the CD4+T cell receptor and co-receptors catalyzing the fusion between the viral envelope and the cell membrane. Most importantly, the env gp160 protein contains epitopes that elicit an immune response in AIDS patients. The REV gene is essential for the processing of the gp160 mRNA and its transport to polysomes.

As shown in FIG. 27, two Amp-6 vectors were prepared—one carrying only the env gene (Amp-6-gp160), and one carrying both the env and REV genes (Amp-6-gp160/REV).

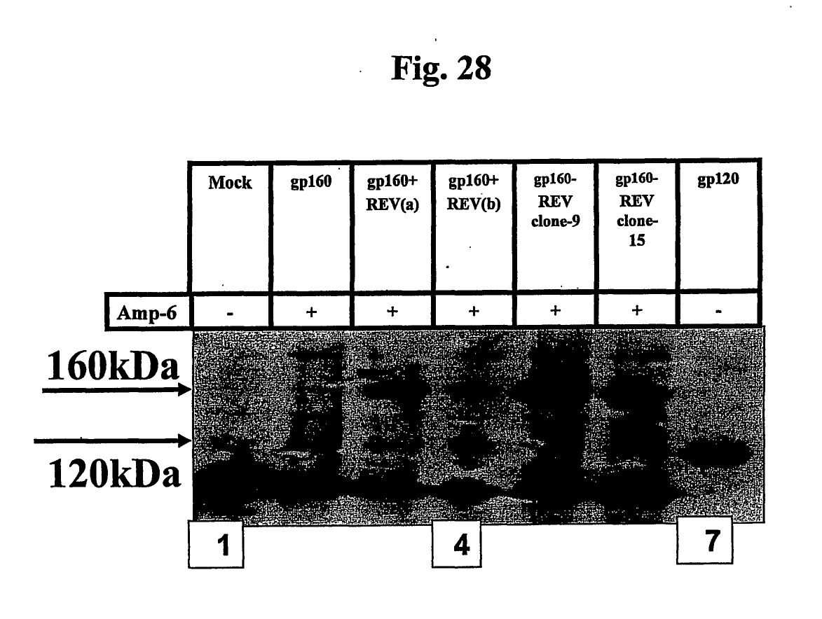

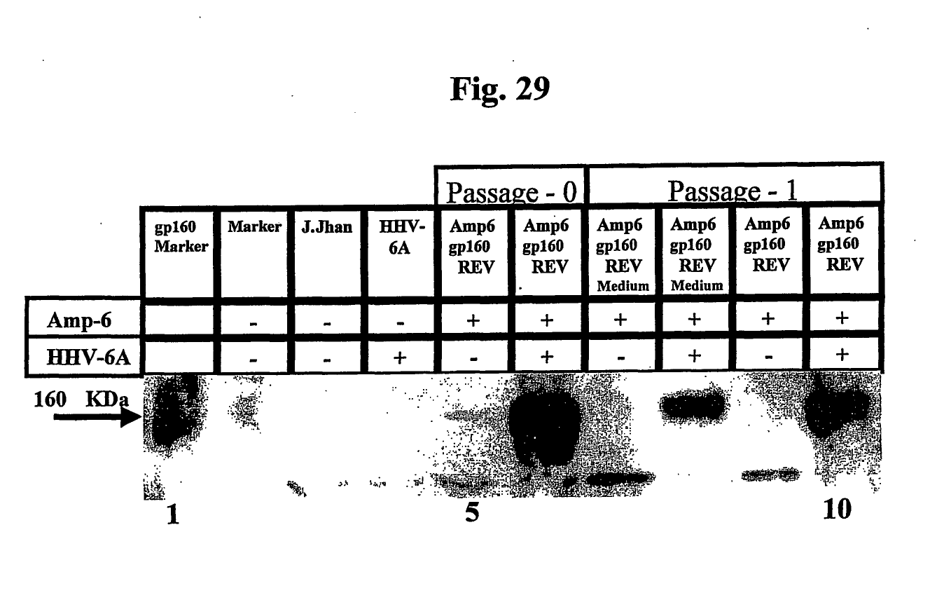

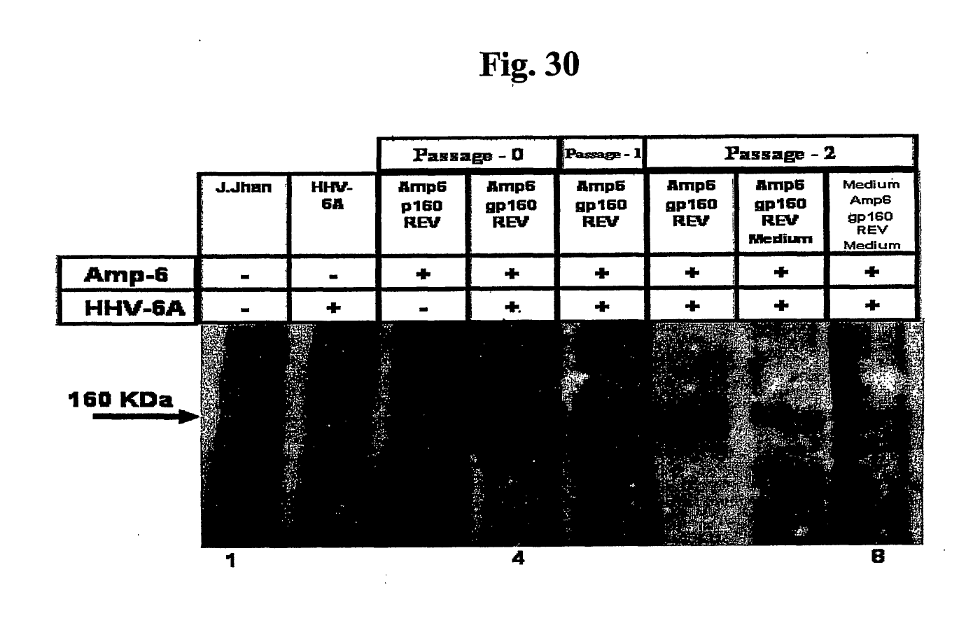

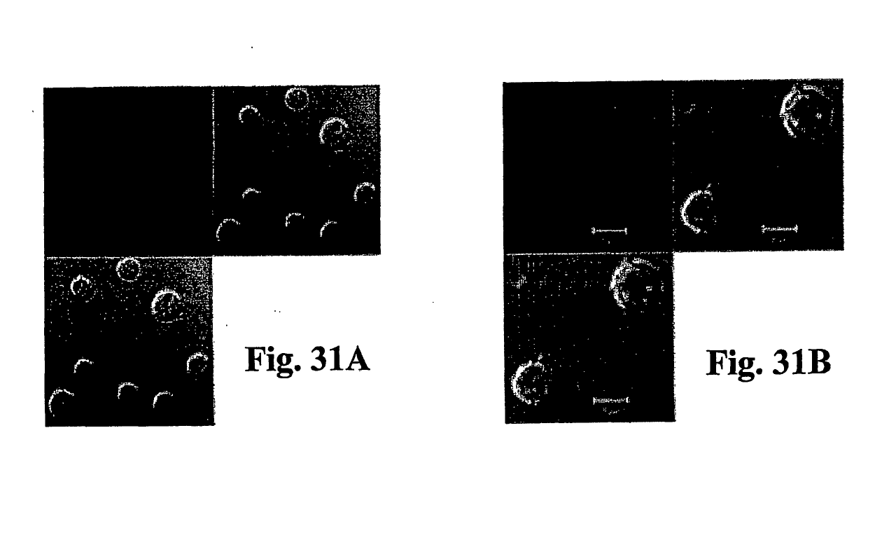

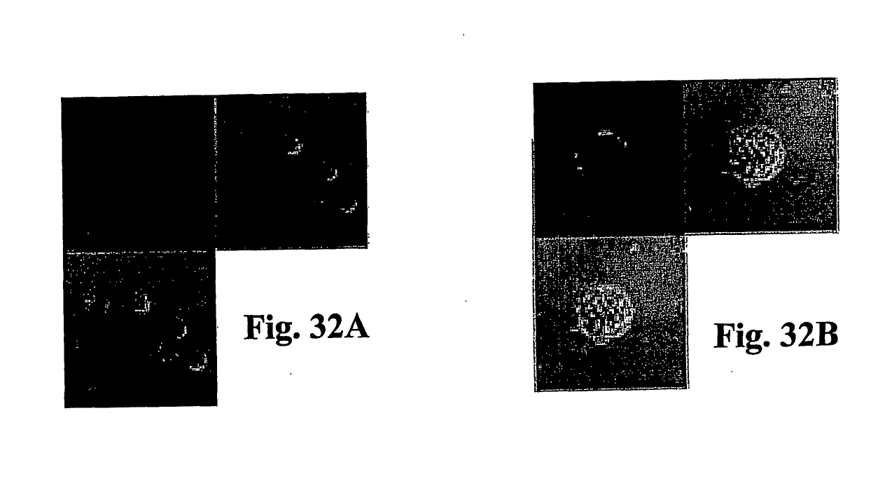

The gp160 protein products and the gp160-REV protein products were found to be expressed in the 293-monolayer cell line when assayed by Western analysis (FIGS. 28 and 29). More importantly the Amp-6-gp160/REV vector could be employed as an infectious virus to T cells, resulting in a very efficient expression of the gp160 gene, as shown by Western blots (FIG. 30). Confocal microscopy (FIGS. 31A and 31B) showed expression of the protein in cell membranes surrounding the cells. Cells transfected Amp6-gp160-EV and superinfected with the helper (FIG. 31B) exhibited fluorescence, whilst the control cells that were infected with the helper alone (FIG. 31A) did not display detectable fluorescence. Similar results with a different antibody are shown in FIGS. 32A and 32B.

Furthermore the gp160/REV amplicon could be further passaged as cell free and cell associated vectors. Expression was significantly enhanced in cells superinfected with the helper virus. It can be concluded that the amplicon and Tamplicon vectors can be efficiently employed to express immunogenic genes in human T cells, including both secreted and membrane-associated proteins.

### Example 11

### Amplicon-6 vector with the MUC1 Sequence

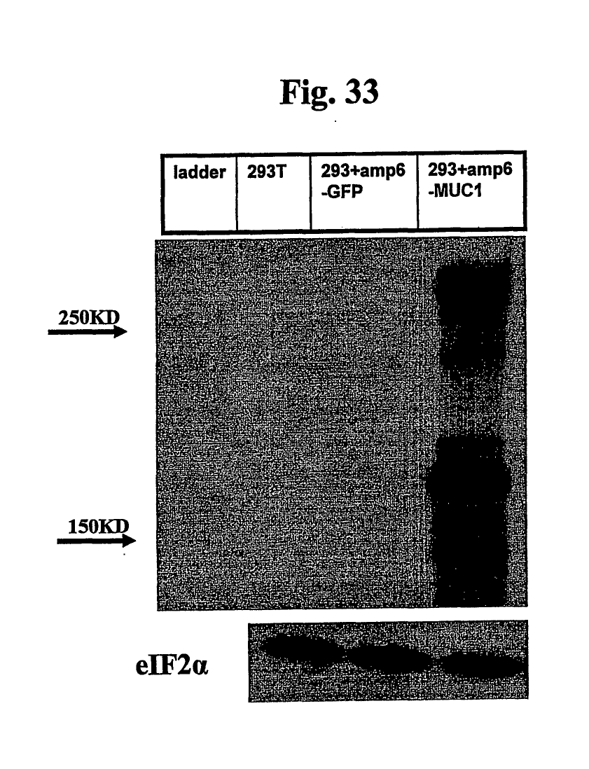

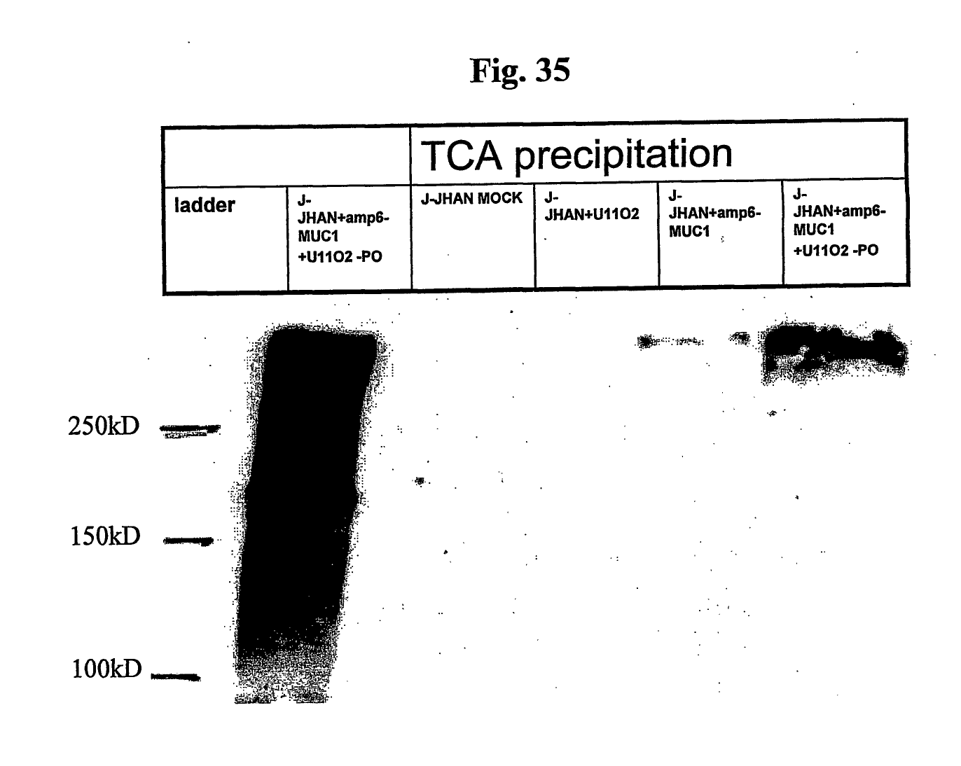

Another nucleotide sequence that was expressed using a vector of the present invention is the MUC1 gene. Amplicon-6-MUC1 vector was transfected in 293T cells. Western blot analysis (FIG. 33) shows that transfection with Amplicon-6-MUC1 caused production of MUC1 proteins (lane 4), whereas no transfection (lane 2) or transfection with a different vector (lane 3) did not provide such proteins. This is also supported by TCA precipitation results shown in FIG. 35, wherein J-JHAN cells that received both amplicon-6-MUC1 and the helper (U1102, lane 6) displayed the most prominent amount of MUC1.

The level of expression of MUC1 protein increased with the addition of a helper virus, as shown in FIG. 41 (compare lanes 4 and 5). This expression was also propagated in passage 1 using helper comprising cells (lane 6).

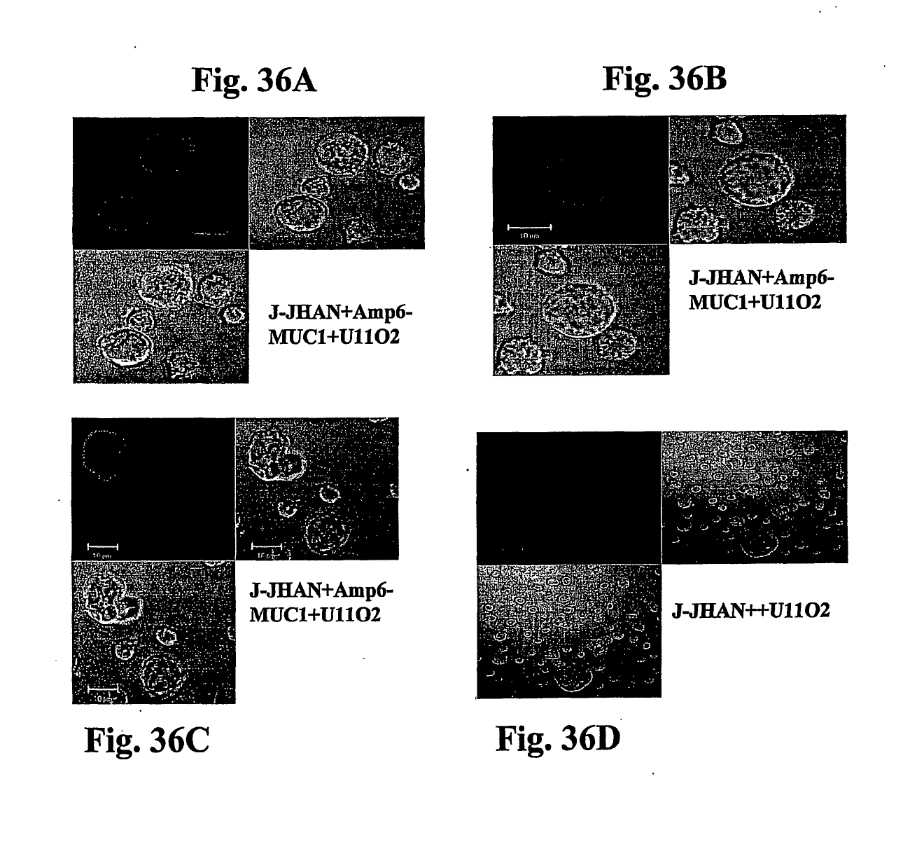

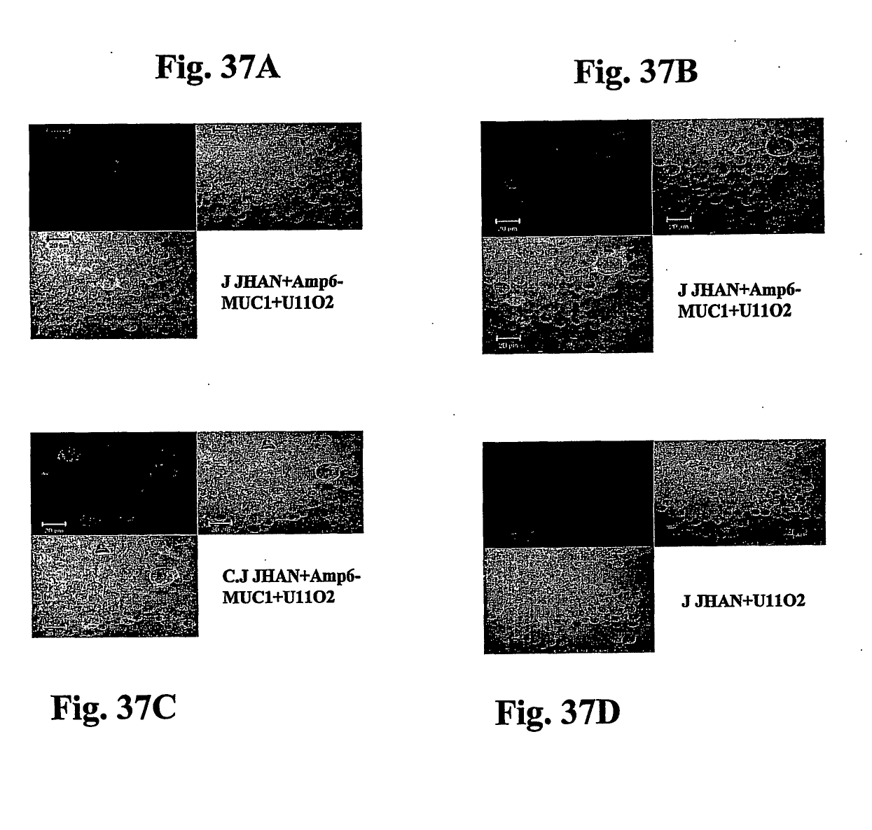

Confocal microscope analyses of amplicon-6-MUC1 infected J-JHAN cells. J-JHAN are shown in FIG. 36A-36D and FIG. 37A-37D. As clearly seen, regardless whether the cells were perforated with triton X100, cells comprising amplicon-6-MUC1 and helper (FIG. 36A-36C and FIG. 37A-37C) displayed fluorescence which was not detected in the control cells that were infected with the helper only (FIG. 36D and 37D).

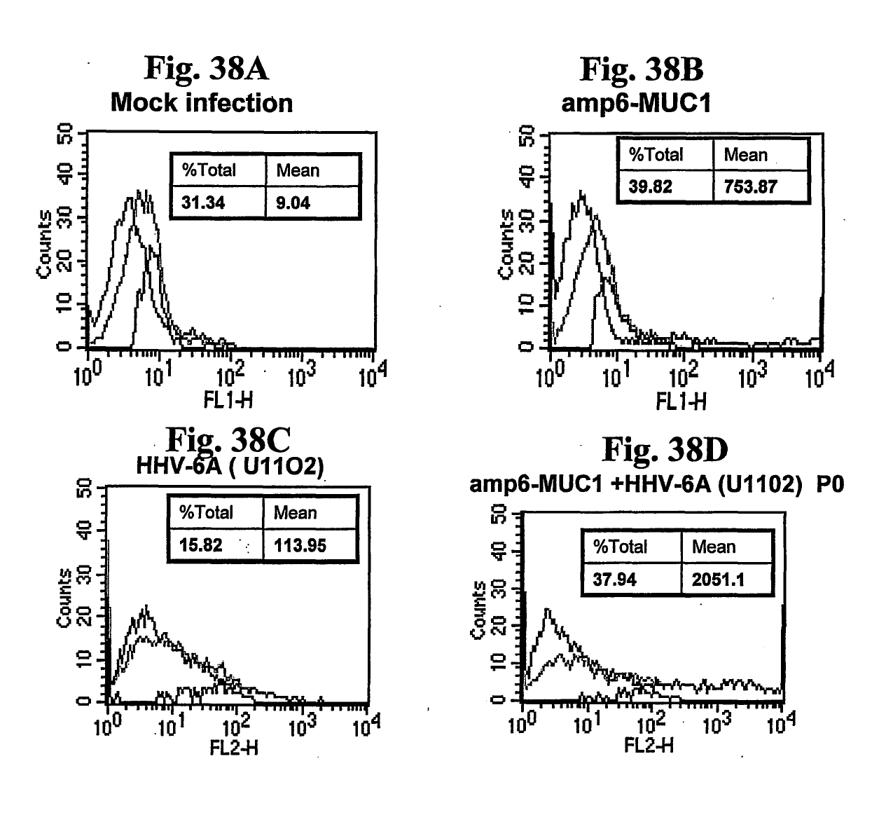

Finally, the above results were also confirmed by FACS (FIG. 38A-38D). Whilst mock infected cells (FIG. 38A) and cells infected with helper only (FIG. 38C) displayed low counts (MFI 9.04 and 113.95, respectively), cells transfected with amplicon-6MUC1 (FIG. 38B) displayed a higher count (MFI 753.87) and the best results were obtained with amplicon-6-MUC1 after superinfection with helper virus (FIG. 38D, MFI 2051.1).

### Example 12

### DNA Vaccination—The Production of Neutralizing Antibodies and the Ability to Induce Cellular Immunization

As a first test of the ability to elicit an immune response using the amplicon-type vectors, DNA vaccination will be utilized. In DNA vaccination, vectors are injected as naked DNA, and not as virion particles. DNA vaccination was shown to result in phagocytosis into macrophages and dendritic cells of the immune system, and in production of neutralizing antibodies as well as induction of CTL activity and secretion of interferons. Thus it is expected that since these cells produce the desired proteins (and even in a manner localized to the membranes) the DNA vaccine is expected to cause the host to produce antibodies. The concatameric nature of the vectors containing multiple repeats of the immunogenic gene is advantageous relative to DNA vaccination with a monomeric plasmid, as is done with other DNA vaccines.

In order to prepare purified amplicon-6 DNA constructs, Amp6-gD, Amp6-gp160/REV gD and Amp-6-MUC1 were purified from total cell DNA by digestion with restriction enzymes which digest the cell DNA and the helper virus DNA into small fragments but do not digest the large (150 kb) concatameric amplicon genomes, which are defective virus genomes. Large amounts of pure defective virus DNA were produced and are being tested in BalbC mice by DNA vaccination done by intradermal (subcutaneous) and intramuscular injections.

Specifically, DNA will be injected into the tail of mice. Testing will be initiated later (e.g. after month). Serum may be tested for the presence of antibodies reactive to the transgene protein by dot blot, by Elisa, as well as neutralizing activity that can reduce viral infectivity several fold, as tested by plaque assays of the virus after it has been exposed to the serum. Injections may be repeated monthly for several months, to boost the response, each time also testing the serum. After several months the mice would be sacrificed and their spleens tested for CD3+CD8⁺ T cells which proliferate and secret γ-interferon and induce cytotoxic in response to the activation with gD.

In all cases described above, the transgenes were driven by the HCMV promoter and are thus expected to be expressed in mouse cells. The amplicon-6 gp160-REV vectors will be tested for the ability to neutralize pseudo HIV replication. The mice will be also tested for the ability to mount immune activity, secretion of interferon and cytolytic activity in response to exposure to the respective antigens The second type of vaccination is with the packaged infectious virus, which was derived ex vivo, in the presence of the helper virus (see below).

It will be readily apparent to the person skilled in the art that the amplicon vectors of the invention could be used for vaccination against many other proteins in addition to those tested. For example, similar use of the amplicon-6 vectors can be done for other herpes viruses utilizing their respective cell surface proteins (e.g., varicella zoster virus, human cytomegalovirus, Epstein Barr virus (EBV) and HHV-8). It can also be done against other (non-herpes) viruses. Additionally, vaccination employing the vector can involve other diseases characterized by defined antigens, such as the MUC1 protein expressed in breast cancer the Prostate Specific Antigen (PSA) which is highly expressed in prostate cancer and HER-2 (neu), which is highly expressed in uterine serious papillary ovarian cancer. The amplicon vectors can be delivered to the T cells and dendritic cells by DNA vaccination and by infection with amplicon virions.

### Example 11

### Vaccination using Amplicon Virions

The second and third types of vaccinations will involve the introduction of cell associated and cell free amplicon-6 vectors carrying the transgene.

For experiments in monkeys, the cell-associated vector will first be derived ex vivo, employing monkey PBMC peripheral blood mononuclear cells). The infected cells will be introduced into the monkey by injection, as described above. Alternatively, filtered, cell-free vectors will be introduced intravenously into Rhesus monkey macaques. The vaccination will be repeated twice to boost the immune response, The serum of vaccinated animals will be tested for production of antibodies by immunoblotting, ELISA, and virus neutralization. Furthermore, CTL assays will be done, testing chromium release.

Effector cells will be prepared by prior incubation with PBMC infected cultures carrying the transgene antigen. The effector cells will be tested for proliferation, secretion of interferons and CTL activity (by chromium release).

For injection into human subjects, packaged amplicon virions are prepared as described in Materials and Methods, and propagated in pathogen-free cells. The vaccine virus may be prepared for vaccination in various ways known in the art.

1-71. (canceled)

72. A vector comprising:
a DNA sequence derived from HHV-6 or HHV-7, said DNA sequence comprising an origin of replication, a cleavage and packaging signal and a promoter sequence which induces expression of at least one nucleic acid sequence product in a lymphocyte cell host;
wherein said vector is adapted to induce an immune response upon administration thereof to a mammal.

73. The vector of claim 72, wherein the vector is replication defective, enabling formation of concatamers of said vector.

74. The vector of claim 72, wherein said DNA sequence is amplicon-6.

75. The vector of claim 72, wherein said DNA sequence is Tamplicon-7.

76. The vector of claim 72, wherein the vector is packaged in a virion particle.

77. The vector of claim 72, wherein, said immune response is elicited against an amino acid product encoded by the DNA sequence of claim 72 or fragments thereof.

78. The vector of claim 72, comprising at least one foreign nucleic acid sequence.

79. The vector of claim 78, wherein at least part of the product expressed by the foreign nucleic acid sequence is targeted to the cell membrane.

80. The vector of claim 79, wherein at least part of the product expressed by the foreign nucleic is secreted outside of the cell.

81. The vector of claim 79, wherein the foreign nucleic acid sequence is selected from sequences coding cellular GFP and B-gal markers, HSV-1 glycoprotein D (gD), gDsec, HIV-1 gp160, REV, tumor antigens, MUC1, Prostate Specific Antigen (PSA), Her-2 (neu) antigen, adjuvant genes, interleukines, cytokines and chemokines.

82. A method for eliciting an immune response in a mammal, said method comprising:
(a) providing a vector of claim 72; and
(b) introducing said vector into the body of said mammal;
(c) wherein said introduction results in an immune response in said mammal.

83. The method of claim 82, further comprising:
(a) providing a helper virus; and
(b) introducing said helper virus into the body of said mammal.

84. The method of claim 83, wherein providing the helper virus is by providing a cell comprising a helper virus.

85. A method according to claim 82, wherein said introduction step (b) comprises:
(a) introducing said vector into lymphotropic cells; and
(b) introducing said lymphotropic cells into said mammal.

86. The method of claim 82, wherein the vector comprises at least one foreign nucleic acid sequence and the immune response is against the protein product encoded by said foreign nucleic acid sequence.

87. The method of claim 85, wherein the lymphotropic cells are selected from dendritic cells (DC), T cells and B cells and any combination thereof.

88. The method of claim 85, further comprising:
(a) providing a helper virus; and
(b) introducing said helper virus into the body of said mammal.

89. Mammalian cells comprising a vector of claim 72.

90. The mammalian cells of claim 89, further comprising a helper virus.

91. The mammalian cells of claim 89, comprising lymphotropic cells selected from dendritic cells (DC), T cells and B cells and any combination thereof.

92. A method of producing mammalian cells capable of producing a product of a nucleic acid sequence of interest, comprising:
(a) providing a vector comprising a foreign nucleic acid sequence of interest according to claim 78;
(b) providing lymphotropic cells that are compatible for transplantation in said mammal; and
(c) introducing said vector to said mammalian cells;
such that said mammalian cells become capable of producing a product of said foreign nucleic acid sequence of interest.

93. The method of claim 92, further comprising:
(a) providing a helper virus; and
(b) introducing said helper virus to said mammalian cells.

94. A method of producing a desired protein comprising:
(a) providing a vector of claim 78, wherein the foreign nucleic acid sequence encodes the desired protein;
(b) providing mammalian cells;
(c) introducing said vector to said mammalian cells; and
(d) providing culture conditions;
such that the mammalian cells produce said desired protein.

95. The method of claim 94, wherein the desired protein is selected from cellular GFP and B-gal markers, HSV-1 glycoprotein D (gD), gDsec, HIV-1 gp160, REV, tumor antigens, MuC1, Prostate Specific Antigen (PSA), Her-2 (neu) antigen, adjuvant genes, interleukines, cytokines and chemokines.

96. The method of claim 94, wherein said mammalian cells are lymphotropic cells.

97. A Concatameric vector comprising repeats of a DNA sequence derived from HHV-6 or HHV-7, said DNA sequence comprising an origin of replication, a cleavage and packaging signal and a promoter sequence which induces expression of at least one nucleic acid sequence product in a lymphocyte cell host, wherein said Concatameric vector is adapted to induce an immune response in a mammal upon administration thereof to said mammal.

98. The Concatameric vector of claim 97, comprising at least one foreign nucleic acid sequence.

99. A method of eliciting an immune response in a mammal comprising administration of the Concatameric vector of claim 97 to said mammal.

100. A method of producing concatameric DNA vectors, comprising:
(a) providing replication defective vector of claim 73;
(b) providing mammalian cells;
(c) introducing said replication defective vector to said mammalian cells; and
(d) providing culture conditions;
such that the mammalian cells produce concatameric DNA vectors.

101. A method of producing virions comprising a vector, said method comprising:
(a) providing a vector of claim 72;
(b) providing mammalian cells;
(c) introducing said vector to said mammalian cells;
(d) providing culture conditions;
such that virions are produced by said mammalian cells.

102. A method for eliciting an immune response in a mammal, said method comprising:
(a) providing a Concatameric vector of claim 97; and
(b) introducing said Concatameric vector into the body of said mammal;
wherein said introduction results in an immune response in said mammal.

103. The method of claim 102, comprising:
(a) providing a helper virus; and
(b) introducing said helper virus into the body of said mammal.

104. A method for eliciting an immune response in a mammal, said method comprising:
(a) providing a virion of claim 76; and
(b) introducing said virion into the body of said mammal;
wherein said introduction results in an immune response in said mammal.

105. A pharmaceutical composition comprising a pharmaceutically acceptable carrier and an effective amount of an active agent selected from:
(a) the vector of claim 72;
(b) the mammalian cells of claim 89;
(c) the Concatameric vector of claim 97; and
(d) the virion of claim 76.


SEQUENCE LISTING (19 sequences)

SEQ ID NO: 1 - DNA, 36 bp, Homo Sapience
cagcttcacgaccggtaggtctcttttgtgtggtgc

SEQ ID NO: 2 - DNA, 36 bp, Homo Sapience
gatactagcctgatcaggggtatctagtaaacaagg

SEQ ID NO: 3 - DNA, 34 bp, Homo Sapience
actagcctgatcactaggcgtcctggatcgacgg

SEQ ID NO: 4 - DNA, 16 bp, Homo Sapience
gaggccccccagattg

SEQ ID NO: 5 - DNA, 17 bp, Homo Sapience
ctgtaagtacgccctcc

SEQ ID NO: 6 - DNA, 18 bp, Homo Sapience
gtaacaactccgccccat

SEQ ID NO: 7 - DNA, 18 bp, Homo Sapience
gtggcaatgagagtgaag

SEQ ID NO: 8 - DNA, 19 bp, Homo Sapience
ctatagcaaagccctttcc

SEQ ID NO: 9 - DNA, 58 bp, Homo Sapience
cagctaccgctggccggccaggcctgtgcagcgtacggtggcaatgagagtgaaggag

SEQ ID NO: 10 - DNA, 59 bp, Homo Sapience
gatactgatcaggccattcaggccttcgaacgtacgctatagcaaagccctttccaaac

SEQ ID NO: 11 - DNA, 19 bp, Homo Sapience
ggagaatagtactaatgcc

SEQ ID NO: 12 - DNA, 19 bp, Homo Sapience
gacaccttaggacagatag

SEQ ID NO: 13 - DNA, 18 bp, Homo Sapience
cagctccaggcaagaatc

SEQ ID NO: 14 - DNA, 18 bp, Homo Sapience
gccctcaagtattggtgg

SEQ ID NO: 15 - DNA, 18 bp, Homo Sapience
ggattgtggaacttctgg

SEQ ID NO: 16 - DNA, 18 bp, Homo Sapience
gcttgatgagtctgactg

SEQ ID NO: 17 - DNA, 16 bp, Homo Sapience
gttcggctgcggcgag

SEQ ID NO: 18 - DNA, 18 bp, Homo Sapience
gtacgcggggctagagcg

SEQ ID NO: 19 - DNA, 18 bp, Homo Sapience
gtaacaactccgccccat